(19) United States
(12) Patent Application Publication
(10) Pub. No.: US 2012/0219535 A1
(43) Pub. Date: Aug. 30, 2012

(54) CRANIAL NEURAL CREST STEM CELLS AND CULTURE CONDITION THAT SUPPORTS THEIR GROWTH

(75) Inventors: Robert E. Maxson, JR., Los Angeles, CA (US); Mamoru Ishii, Los Angeles, CA (US)

(73) Assignee: University of Southern California

(21) Appl. No.: 13/499,664

(22) PCT Filed: Aug. 31, 2010
(86) PCT No.: PCT/US10/47337

§ 371 Date: May 10, 2012

Related U.S. Application Data
(60) Provisional application No. 61/248,302, filed on Oct. 2, 2009.
(60) Provisional application No. 61/322,742, filed on Apr. 9, 2010.

(51) Int. Cl. A61K 35/12; C12Q 1/02; C12N 5/0797; A61P 25/00; G01N 33/566; A61P 19/08; A61P 19/02; A61P 19/00; C12N 5/0735; C12Q 1/68

(52) U.S. Cl. 424/93.7; 435/325; 435/29; 435/405; 435/6.12; 435/6.1; 435/7.21

(57) ABSTRACT

Provided herein is a method to isolate a cranial neural crest stem cell and novel compositions containing the cell. Also provided are compositions and methods to clonally expand the population and differentiate the cells into various phenotypes. Therapeutic methods for the compositions are further provided.

### CROSS-REFERENCE TO RELATED APPLICATIONS

This application claims the benefit under 35 U.S.C. §119(e) of U.S. Provisional Application Nos. 61/248,302 and 61/322,742, filed Oct. 2, 2009 and Apr. 9, 2010, respectively. The contents of these applications are hereby incorporated by reference in their entirety.

### BACKGROUND

Throughout this disclosure, various technical and patent publications are referenced to more fully describe the state of the art to which this invention pertains. Some of the references are identified by first author name and date of publication. The full bibliographic information for these publications can be found at the end of the specification, immediately preceding the claims. These publications are incorporated by reference, in their entirety, into this application.

Accidental injuries, diseases resulting in tissue degeneration, congenital disorders, and surgical treatments of tumors all can produce severe deficiencies in craniofacial organs and tissues. Among the tissues affected by such pathological processes are the skeleton, cartilage, joints, muscles, connective tissues, adipogenic tissues, and sensory organs.

Anomalies in neural crest stem cells during embryonic development are a potential cause of the human congenital disorder, Hirschsprung disease, in which failure of trunk neural crest stem cell migration during gut development causes a defect in enteric nerve innervation (Iwashita et al. (2003)). It is possible that defects of stem cells in the cranial neural crest also cause pathological conditions in craniofacial development.

Craniofacial defects can have profound physical and psychological impacts on the quality of life of affected individuals. Thus, appropriate surgical treatment is vital for the reconstruction of these defects. Despite advances in tissue engineering technology, reconstructive surgery often results in suboptimal outcomes. Improvement in approaches to the repair and regeneration of craniofacial tissues has become a major goal. One category of defect that is both difficult to treat and a source of significant morbidity is a critical size bone defect. Such defects consist of bone lesions that are so large as to preclude healing without some form of grafting.

In reconstructive surgeries performed on cranial tissue, autogenous grafts appear to have a better prognosis than allogeneic grafts from non-craniofacial tissues (D'Addona and Nowzari (2001)). Recent work has shown that this may be a result of differences in cellular and molecular identities between craniofacial and non-craniofacial tissues (Leucht et al. (2008)). Thus, autogenous implantation is the favored approach to treat defects in the cranial apparatus. Another recent finding has shown the feasibility of using embryonic mandibular neural crest to repair defects in the cranial skeleton (Chung et al. (2009)). Stable production of biomaterial derived from cranial neural crest may promote optimal strategies for cranial tissue reconstruction. However, it is not clear how stably these stem-like cells can be maintained in culture over the long-term (Chung et al. (2009)). Limitations in the supply of craniofacial tissues has been a major impediment to autogenous implantation.

Therefore, identification of multipotent neural crest stem cells and establishing a protocol for culturing them will provide insight into fundamental mechanisms of cranial neural crest development and will aid in the understanding of congenital human diseases.

A cell culture system would be a powerful asset for the investigation of the development of cranial neural crest. One of the biggest challenges in the field is to establish a model that will enable genetic manipulation and mass biochemical analysis in vitro. Such a model must have the capability of providing a large quantity of homogeneous cells that represent the native status of cranial neural crest cells. Currently, there is no generally accepted sustainable cell culture model for cranial neural crest. Cranial neural crest stem cells could be an ideal reagent for this.

The identification of expandable, multipotent cell populations in the cranial neural crest and the establishment of a protocol for culturing them will pave the way for practical cell-based therapy of the craniofacial tissue reconstitution. However, previous studies have suggested that although multipotent stem-like cells may exist in the developing cranial neural crest, they are transient, undergoing lineage restriction early in embryonic development (Baroffio et al. (1988), Trentin et al. (2004)). Support for this view, largely negative, comes from the finding that the stem cell status of cranial neural crest has never been maintained in vitro.

The neural crest, a population of multipotent, migratory cells, plays a variety of crucial roles in vertebrate organogenesis (Chai and Maxson (2006)). Neural crest cells are specified at the border between neural and non-neural ectoderm during embryogenesis. After specification, they undergo an epithelial-mesenchymal transition and migrate into the ventro-lateral aspect of the embryo where they form different cell-types and organs. Depending on the site of origin along the anterior-posterior axis of the embryonic body, neural crest cells are sub-categorized into cranial, cardiac, and trunk populations. Each group has a unique developmental potential. The cranial neural crest, which originates in the portion of the neural tube from the neural fold anterior to rhombomere 6, has the ability to produce a greater diversity of derivatives than other crest populations: Cranial neural crest cells give rise to cranial skeletal bone, cartilage, dentin, smooth muscle, adipogenic tissues, melanocytes, corneal endothelial cells, and peripheral nerves. Trunk neural crest cells form a more limited set of cell types, including peripheral nerves, melanocyte, and adrenal medulla (Santagati and Rijli (2003)).

Although the multipotency of single cranial neural crest cells has been reported, the ability of these stem-like cells to self-renew has so far been a matter of conjecture. An experiment conducted by Le Douarin's group in 1988 showed that when single quail cranial neural crest cells were co-cultured with growth inhibited Swiss 3T3 cells, they produced neurons, melanocytes, and non-neuronal cells in vitro (Baroffio et al. (1988)). A recent study using similar culture conditions demonstrated that a cranial neural crest clone is capable of producing six different cell-types (osteoblast, chondrocyte, myofibroblast, melanoblast, glia, and neuron (Calloni et al. (2007), Calloni et al (2009)). Thus, the multipotency of single cranial neural crest cells is evident. However, cells used in these co-culture experiments were transient—i.e., were not maintained as cell lines. Whether these clones had the ability to self-renew was not determined.

Dupin's group has sought to address this issue using an approach that did not involve co-culturing of neural crest cells. Instead, they used culture dishes coated with collagen and a medium containing 2% chicken embryonic extract and 10% FCS with or without endothelin-3 (Trentin et al. (2004)). Seven passageable individual clones were established and were assessed for the extent to which they were multiripotent. The results suggested that the clones with the ability to self-renew were not multipotent stem cells, but lineage-restricted bipotent (glia-myofibroblast, or glia-melanoblast) or unipotent progenitors (Trentin et al. (2004)). Thus, Dupin and colleagues concluded that multipotent stem-like cells in cranial neural crest undergo progressive lineage restriction and that heterogeneous progenitors with limited potency serve as a source of terminally differentiated cells during vertebrate craniofacial organogenesis.

In contradistinction to this finding, self-renewing multipotent stem cells have been reported in another neural crest population—the trunk neural crest. Trunk neural crest cells isolated from E10.5 rat embryos contain a group of cells that express p75 (NGFR) and nestin. These cells can be propagated on fibronectin-coated culture dishes in medium supplemented with chicken embryonic extract, bFGF, and EGF. Clones derived from these cells can produce subclones and maintain the ability to become neurons, glial cells, and smooth muscle cells. Thus, trunk neural crest may contain a population of multipotent stem cells that self-renew (Stemple and Anderson (1992)). The sciatic nerve, a trunk neural crest derivative, also produces a similar stem cell population in late gestation (E17.5 rat (Morrison et al. (1999)). It is possible that trunk neural crest stem cells serve as source of neurons and glia through sciatic nerve development. Self-renewing, multipotent stem cells also have been found in neural crest-derived, postnatal adult tissues including the hair follicle (SKPs and EPI-NCSCs), cornea (COPs), cardiac tissues, enteric nerve, and carotid body (Delfino-Machin (2007); Pardal (2007)). Cranial neural crest progenitors with a capacity to self renew had limited potency when compared with their counterparts in the trunk neural crest.

Therefore, there is a need for identification of expandable, self-renewable and multipotent stem cells in the cranial neural crest and the establishment of a protocol for culturing them for cell-based therapy.

### SUMMARY OF THE INVENTION

The identification of expandable, self-renewable stem cells in the cranial neural crest and the establishment of a protocol for culturing them will pave the way for practical cell-based therapy of the craniofacial tissue reconstitution.

The current invention provides an isolated self-renewable cranial neural crest stem cell and a clonal population of the stem cell that are useful in such therapies. The self-renewable cranial neural crest stem cell or clone is multipotent can be isolated from mammalian embryo, embryonic stem (ES) cells, non-fetal tissue or induced pluripotent stem cells. In some embodiments, these multipotent stem cells are capable of differentiating into at least one, or alternatively at least two, or alternatively at least three, or alternatively at least four cell, or alternatively at least five, or alternatively at least six types selected from the group of an osteoblast, a chondrocyte, a smooth muscle cell a glial cell, a neuronal cell or an adipocyte.

Also provided is an isolated population of self-renewable multipotent cranial neural crest stem cells. In some embodiments, the isolated population of self-renewable multipotent cranial neural crest stem cells is substantially homogenous.

The invention further provides a neural crest stem cell growth medium comprising Dulbecco's modified Eagle's medium (DMEM) and fetal bovine serum (FBS). In one aspect, the medium further comprises MEM nonessential amino acids, sodium pyruvate, β-mercaptoethanol, penicillin, streptomycin and L-glutamine. In another aspect, the medium is conditioned by STO feeder cells. In yet another aspect, the medium is supplemented with basic fibroblast growth factor (bFGF) and/or leukemia inhibitory factor (LIF).

Methods of isolating, preparing, culturing, expanding, propagating and/or differentiating the stem cells, and methods of using the cells or populations to treat diseases are also disclosed in the current invention.

### BRIEF DESCRIPTION OF THE FIGURES

FIGS. 1A-L show sustainable stem-like potency of mass cultured cranial neural crest. (A) Overall strategy. Cranial neural crest cells labeled with Wnt1-Cre; R26R-EGFP were obtained from E8.5 mouse embryos. Dissociated cells were initially expanded in vitro for 3 days and FACS sorted (arrowhead). Sorted cells were cultured on Matrigel coated plates with basal medium. Under this condition, cranial neural crest cells can be passaged for an extended time. Two independent mass culture lines were established (O9-1 and i10-1). (B,C) The morphology (B) and growth ratio (C) of mass culture #i10-1. Doubling time is approximately four days. (D-I) Long-term cultured mass cranial neural crest differentiated into multiple cell-lineages. Shown is line i10-1, which was capable of differentiating into osteoblasts (D), chondrocytes (E), smooth muscle cells (F), adipocytes (G), and glial cells (H). (I) Marker gene expression analysis by RT-PCR. Both lines (O9-1/passage11 and i10-1/passage10) expressed AP-2α, Twist, and Snail1 (neural crest markers). They also expressed nestin, CD44, Sca-1 (stem cell markers). (J) Flow cytometry analysis of i10-1 for CD44 and Sca-1 expression. More than 97% of i10-1 cells were double positive for CD44 and Sca-1 (right). Isotype antibodies (negative control; left and middle) showed no staining (K,L) Endogenous expression of CD44 in E8.0 Wnt1-Cre;R26R-EGFP embryo. The majority of migrating cranial neural crest cells (EGFP; dark gray) were positive for CD44 (PE; gray). Enlarged view of boxed area in K is shown in L. (L) CD44 positive cranial neural crest cells are indicated by arrows.

FIG. 2A-C illustrate strategy for cranial neural crest clonal culture. (A) Cranial neural crest cells marked with Wnt1-Cre;R26R-EGFP were obtained from E8.5 mouse embryos. Dissociated cells were initially expanded in vitro for 3 days and FACS sorted (arrowhead). 288 EGFP positive cells were clonally seeded on three 96 wells plates (a single cell per well) by means of an automated cell-seeding device. Initially, single cells were co-cultured with growth-inhibited STO feeder cells (10³ cells/cm²) in basal medium. After three weeks, wells seeded with control feeder cells had no obvious cellular growth (B), while nine cranial neural crest-seeded wells had colonies of cells. An example of a primary colony is shown in (C). These cells were trypsinized and passaged on Matrigel or fibronectin coated plates (without a feeder layer). Among them, 3 lines were passageable clones (C7-3, C7-8, and D7-1). Thus, the plating efficiency of clonal culture was 1.04%.

FIG. 3A-T show differentiation potential of cranial neural crest clones. (A-M) The morphology, cell growth, and differentiation potency of clone #C7-8. (A,B) The appearance (A) and cell growth (B) of clone #C7-8. This clone's doubling time is approximately 10 days. (C,D) In vitro differentiation assay of C7-8. (C) Cells treated with osteogenic medium for 3 weeks express ALP (alkaline phosphatase), an early osteoblast differentiation marker. (D) C7-8 also produced smooth muscle cells when cultured in TGF-13 supplemented medium for 3 weeks (red; αSMA). (E-M) We tested the differentiation potential of C7-8 in vivo. (E) Schematic drawing of exo utero microinjection experiments. EGFP expressing C7-8 cells (round) were microinjected into the frontal bone primordium (bpd; blue) of E13.5 mouse embryos (bh; brain hemisphere, e; eye). (F) A coronal section of E13.5 control embryo (Ohr) shows microinjected C7-8 cells (red, arrowheads) at the site of injection (is). EGFP expression of injected cell was immunohistochemically detected with DAB staining (G,H,I) Embryo at 72 hrs after injection. (G) C7-8 cells (arrows) have migrated toward distal area of developing calvarial bone along with host osteoprogenitors. At this stage, as expected from our work on osteoprogenitor migration (Ting et al. (2009)), the injected cells are not found in the calvarial bone osteomatrix labeled by ALP, but are in the process of migrating in a cell layer located outside (ectocranial) to the developing bone (H). (I) Approximate location of injected cells is indicated in the scheme. (J,K,L,M) Embryo at 5 days after injection. (J,K) Injected cells have integrated into mineralized calvarial bone (bp) at distal location, consistent with the normal behavior of osteoprogenitors. The adjacent section was stained for ALP expression to detect osteoblasts (L) (sk; skin) (M) Approximate location of injected cells. (N-S) Preliminary analysis of cranial neural crest clone #D7-1. (N,O,P) Morphology (N), cell growth (O), and EGFP expression (P) of clone #D7-1. This clone's doubling time is approximately 3 days. (Q,R,S,T) Intriguingly, D7-1 cells are capable of differentiating into osteoblasts (Q), chondrocytes (R), smooth muscle cells (S), and glial cells (T (light gray; GFAP)).

FIG. 4A-B shows expression of Sca-1 is characteristic to undifferentiated state of craNCSC clone D7-1. (A) 24hrs hanging-drop culture induces osteogenic differentiation of D7-1. Cells were harvested at 6 hrs, 12 hrs, and 24 hrs of culture period and stained with alizarin red. Profound osteogenic differentiation was evident in 24 hrs, but not 6 hrs cultured hanging-drop. (B) Gene expression analysis of markers for the stem cell and osteogenic differentiation. RNA was extracted from hanging-drops culture at each time point and subjected to RT-PCR analysis of Sca-1 (stem cell), Msx2 and Runx2 (osteoprogenitor), ALP and Osteocalcin (terminally differentiated osteoblast). The intensity of PCR products was quantified by NIH ImageJ after gel running and normalized by β-actin. A dramatic reduction of Sca-1 expression within 24 hrs. This expression pattern is complement to transient up-regulation of Msx2 and Runx2, as well as induction of ALP and Osteocalcin expression.

FIG. 5A-G shows undifferentiated craNCSC marker CD93 is expressed in subpopulation of migratory mouse cranial neural crest. (A and B) RT-PCR analysis of CD93 expression in proliferative and differentiated D7-1. (A), RT-PCR results show a expression level of CD93 in D7-1 cells was greatly reduced when cells differentiated into osteogenic-lineage cells. (B), PCR product was quantified and normalized by β-actin. CD93 expression was reduced in differentiated D7-1 by 72%. Thus, it serves as a marker of undifferentiated craNCSC. (C-G) CD93 expression in mouse cranial neural crest cells. (C, D, E), Transverse cryosection of E8.5 Wnt1-Cre; R26R; EGFP embryo was stained with PE-conjugated anti-CD93 antibody. Cranial neural crest (green) expresses CD93 (red) in a part of its migratory population (arrows). Enlarged views of boxed areas in C are shown in D and E. (F and G), CD93 expression in mouse cranial tissue at E9.5. CD93 expression in a migratory cranial neural crest had become more restricted at E9.5 than E8.5. This suggests CD93 is only expressed in immature cranial neural crest stem cell. Boxed area in F is shown at higher magnification in G. Abbreviations, ne; neural ectoderm, nt; neural tube.

FIG. 6A-C shows gutNCSC and craNCSC are not same, but related subpopulation of the neural crest cell. (A-C) Expression analysis of gutNCSC markers in craNCSC. (A), RT-PCR assay for gutNCSC markers in whole E8.5 mouse embryos (E8.5) and craNCSC clones, D7-1, N16-1, and N16-16. (B), Quantified RT-PCR results. Relative values of expression to whole E8.5 are shown. Some gutNCSC markers, CD9 and Gfra1, are expressed prominently in craNCSC while others, Ret, Sox10, Ednrb, and Gas7, are not. (C), Additional expression analysis of trunkNCSC marker in D7-1. Data from whole genome transcriptome of D7-1 show that conventional trunk neural crest stem cell marker p75 and Intga4 are not robustly expressed in craNCSC clone D7-1. On the other hand, nestin, another trunkNCSC maker, is highly expressed in D7-1. These results illuminate remarkable similarity and dissimilarity in gutNCSC and craNCSC.

FIGS. 7A and B shows that AG490 treatment causes severe cell mortality in both primary cultured and long-term cultured craNCSC. (A), AG490 treatment for long-term cultured craNCSC clone D7-1. Cells were treated either basal medium with 0.4% DMSO (vehicle control) or AG490 at the dose of 10 μM or 20 μM for three days. Alive cell number was counted daily. 20 μM AG490 treatment caused a significant depletion of cell survival while 10 μM treatment had more modest effect. (B), AG490 treatment for primary cultured cranial neural crest. Cranial neural crest cells labeled with Wnt1-Cre;R26R-EGFP were FACS sorted from E8.5 mouse embryos. Then, they were immediately cultured in basal medium either with DMSO or AG490 (10 μM or 20 μM) for three days. The number of alive cell was counted. High cell mortality which we have seen in AG490 treated D7-1 was also evident in those cells.

### DETAILED DESCRIPTION OF THE INVENTION

### Definitions

The practice of the present invention will employ, unless otherwise indicated, conventional techniques of tissue culture, immunology, molecular biology, microbiology, cell biology and recombinant DNA, which are within the skill of the art. See, e.g., Sambrook, Fritsch and Maniatis (1989) Molecular Cloning: A Laboratory Manual, 2^{nd} edition; F. M. Ausubel, et al. eds. (1987) Current Protocols In Molecular Biology; the series Methods in Enzymology (Academic Press, Inc.): PCR 2: A Practical Approach (1995) (M.J. MacPherson, B. D. Hames and G. R. Taylor eds.); Harlow and Lane, eds. (1988) Antibodies, A Laboratory Manual; Harlow and Lane, eds. (1999) Using Antibodies, a Laboratory Manual; and R. I. Freshney, ed. (1987) Animal Cell Culture.

All numerical designations, e.g., pH, temperature, time, concentration, and molecular weight, including ranges, are approximations which are varied (+) or (−) by increments of 1.0 or 0.1, as appropriate. It is to be understood, although not always explicitly stated that all numerical designations are preceded by the term “about”. It also is to be understood, although not always explicitly stated, that the reagents described herein are merely exemplary and that equivalents of such are known in the art.

As used in the specification and claims, the singular form “a,” “an” and “the” include plural references unless the context clearly dictates otherwise.

As used herein, the term “comprising” is intended to mean that the compositions and methods include the recited elements, but do not exclude others. “Consisting essentially of” when used to define compositions and methods, shall mean excluding other elements of any essential significance to the combination when used for the intended purpose. Thus, a composition consisting essentially of the elements as defined herein would not exclude trace contaminants or inert carriers. “Consisting of” shall mean excluding more than trace elements of other ingredients and substantial method steps. Embodiments defined by each of these transition terms are within the scope of this invention.

The term “isolated” as used herein refers to molecules or biological or cellular materials being substantially free from other cellular materials present in the native environment, e.g., greater than 70%, or 80%, or 85%, or 90%, or 95%, or 98%. In one aspect, the term “isolated” refers to nucleic acid, such as DNA or RNA, or protein or polypeptide, or cell or cellular organelle, or tissue or organ, separated from other DNAs or RNAs, or proteins or polypeptides, or cells or cellular organelles, or tissues or organs, respectively, that are present in the natural source and which allow the manipulation of the material to achieve results not achievable where present in its native or natural state, e.g., recombinant replication or manipulation by mutation. The term “isolated” also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides, e.g., with a purity greater than 70%, or 80%, or 85%, or 90%, or 95%, or 98%. The term “isolated” is also used herein to refer to cells or tissues that are isolated from other cells or tissues and is meant to encompass both cultured and engineered cells or tissues.

As used herein, “stem cell” defines a cell with the ability to divide for indefinite periods in culture and give rise to specialized cells. At this time and for convenience, stem cells are categorized as somatic (adult), embryonic, cells and/or parthenogenetic stem cells (see Cibelli et al. (2002) Science 295(5556):819; U.S. Patent Publ. Nos. 20100069251 and 20080299091), or induced pluripotent stem cells (iPS or iPSC). A somatic stem cell is an undifferentiated cell found in a differentiated tissue that can renew itself (clonal) and (with certain limitations) differentiate to yield all the specialized cell types of the tissue from which it originated. An embryonic stem cell is a primitive (undifferentiated) cell from the embryo that has the potential to become a wide variety of specialized cell types. Non-limiting examples of embryonic stem cells are the HES2 (also known as ES02) cell line available from ESI, Singapore and the H1 or H9 (also know as WA01) cell line available from WiCell, Madison, Wis. Additional lines are available from the NIH and commercial vendors. See for examplegrants.nih.gov/stem cells/registry/current.htm (last accessed Oct. 2, 2009). Pluripotent embryonic stem cells can be distinguished from other types of cells by the use of markers including, but not limited to, Oct-4, alkaline phosphatase, CD30, TDGF-1, GCTM-2, Genesis, Germ cell nuclear factor, SSEA1, SSEA3, and SSEA4. An induced pluripotent stem cell (iPSC) is an artificially derived stem cell from a non-pluripotent cell, typically an adult somatic cell, produced by inducing expression of one or more stem cell specific genes.

“Embryoid bodies or EBs” are three-dimensional (3-D) aggregates of embryonic stem cells formed during culture that facilitate subsequent differentiation. When grown in suspension culture, EBs cells form small aggregates of cells surrounded by an outer layer of visceral endoderm. Upon growth and differentiation, EBs develop into cystic embryoid bodies with fluid-filled cavities and an inner layer of ectoderm-like cells.

The term “propagate” means to grow or alter the phenotype of a cell or population of cells. The term “growing” refers to the proliferation of cells in the presence of supporting media, nutrients, growth factors, support cells, or any chemical or biological compound necessary for obtaining the desired number of cells or cell type. In one embodiment, the growing of cells results in the regeneration of tissue. In yet another embodiment, the tissue is comprised of neuronal progenitor cells or neuronal cells.

The term “culturing” refers to the in vitro propagation of cells or organisms on or in media of various kinds It is understood that the descendants of a cell grown in culture may not be completely identical (i.e., morphologically, genetically, or phenotypically) to the parent cell. By “expanded” is meant any proliferation or division of cells.

As used herein and as set forth in more detail below, “conditioned medium” is medium which was cultured with a mature cell that provides cellular factors to the medium such as cytokines, growth factors, hormones, and extracellular matrix.

“Differentiation” describes the process whereby an unspecialized cell acquires the features of a specialized cell such as a heart, liver, or muscle cell. “Directed differentiation” refers to the manipulation of stem cell culture conditions to induce differentiation into a particular cell type. “Dedifferentiated” defines a cell that reverts to a less committed position within the lineage of a cell. As used herein, the term “differentiates or differentiated” defines a cell that takes on a more committed (“differentiated”) position within the lineage of a cell. As used herein, “a cell that differentiates into a mesodermal (or ectodermal or endodermal) lineage” defines a cell that becomes committed to a specific mesodermal, ectodermal or endodermal lineage, respectively. Examples of cells that differentiate into a mesodermal lineage or give rise to specific mesodermal cells include, but are not limited to, cells that are adipogenic, leiomyogenic, chondrogenic, cardiogenic, dermatogenic, hematopoetic, hemangiogenic, myogenic, nephrogenic, urogenitogenic, osteogenic, pericardiogenic, or stromal.

Examples of cells that differentiate into ectodermal lineage include, but are not limited to epidermal cells, neurogenic cells, and neurogliagenic cells.

As used herein, the term “differentiates or differentiated” defines a cell that takes on a more committed (“differentiated”) position within the lineage of a cell. “Dedifferentiated” defines a cell that reverts to a less committed position within the lineage of a cell.

As used herein, a “pluripotent cell” defines a less differentiated cell that can give rise to at least two distinct (genotypically and/or phenotypically) further differentiated progeny cells. In another aspect, a “pluripotent cell” includes an induced Pluripotent Stem Cell (iPSC) which is an artificially derived stem cell from a non-pluripotent cell, typically an adult somatic cell, produced by inducing expression of one or more stem cell specific genes. Such stem cell specific genes include, but are not limited to, the family of octamer transcription factors, i.e. Oct-3/4; the family of Sox genes, i.e. Sox1, Sox2, Sox3, Sox 15 and Sox 18; the family of Klf genes, i.e. Klf1, Klf2, Klf4 and Klf5; the family of Myc genes, i.e. c-myc and L-myc; the family of Nanog genes, i.e. OCT4, NANOG and REX1; or LIN28. Examples of iPSCs are described in Takahashi et al. (2007) Cell advance online publication 20 Nov. 2007; Takahashi & Yamanaka (2006) Cell 126:663-76; Okita et al. (2007) Nature 448:260-262; Yu et al. (2007) Science advance online publication 20 Nov. 2007; and Nakagawa et al. (2007) Nat. Biotechnol. Advance online publication 30 Nov. 2007.

A “multi-lineage stem cell” or “multipotent stem cell” refers to a stem cell that reproduces itself and at least two further differentiated progeny cells from distinct developmental lineages. The lineages can be from the same germ layer (i.e. mesoderm, ectoderm or endoderm), or from different germ layers. An example of two progeny cells with distinct developmental lineages from differentiation of a multilineage stem cell is a myogenic cell and an adipogenic cell (both are of mesodermal origin, yet give rise to different tissues). Another example is a neurogenic cell (of ectodermal origin) and adipogenic cell (of mesodermal origin).

“Self-renewable” refers to a cell being able to self-renew for over a number of passages without substantial changes of cell properties. In one aspect, the number of passages is at least about 5, or alternatively at least 10, or alternatively at least about 15, 20, 30, 50, or 100.

As used herein, the “lineage” of a cell defines the heredity of the cell, i.e. its predecessors and progeny. The lineage of a cell places the cell within a hereditary scheme of development and differentiation.

As used herein, the term “differentiates or differentiated” defines a cell that takes on a more committed (“differentiated”) position within the lineage of a cell. “Dedifferentiated” defines a cell that reverts to a less committed position within the lineage of a cell.

As used herein, “a cell that differentiates into a mesodermal (or ectodermal or endodermal) lineage” defines a cell that becomes committed to a specific mesodermal, ectodermal or endodermal lineage, respectively. Examples of cells that differentiate into a mesodermal lineage or give rise to specific mesodermal cells include, but are not limited to, cells that are adipogenic, leiomyogenic, chondrogenic, cardiogenic, dermatogenic, hematopoetic, hemangiogenic, myogenic, nephrogenic, urogenitogenic, osteogenic, pericardiogenic, or stromal.

Clonal and subclonal population of cells are cells that maintain the original phenotypic markers and multipotency as the parent cell from which is was reproduced.

A “clonal culture” is a group of cells originated from one ancestor cell. Subclonal culture is a group of cells originated from one of clonally cultured cell. By comparing parental clonal and descendant subclonal culture, one should be able to determine whether subclonal population maintain the original phenotypic markers and multipotency.

A “composition” is also intended to encompass a combination of active agent and another carrier, e.g., compound or composition, inert (for example, a detectable agent or label) or active, such as an adjuvant, diluent, binder, stabilizer, buffers, salts, lipophilic solvents, preservative, adjuvant or the like. Carriers also include pharmaceutical excipients and additives proteins, peptides, amino acids, lipids, and carbohydrates (e.g., sugars, including monosaccharides, di-, tri-, tetra-, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid/antibody components, which can also function in a buffering capacity, include alanine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this invention, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

“Substantially homogeneous” describes a population of cells in which more than about 50%, or alternatively more than about 60%, or alternatively more than 70%, or alternatively more than 75%, or alternatively more than 80%, or alternatively more than 85%, or alternatively more than 90%, or alternatively, more than 95%, of the cells are of the same or similar phenotype. Phenotype can be determined by a pre-selected cell surface marker or other marker.

A neuron is an excitable cell in the nervous system that processes and transmits information by electrochemical signaling. Neurons are found in the brain, the vertebrate spinal cord, the invertebrate ventral nerve cord and the peripheral nerves. Neurons can be identified by a number of markers that are listed on-line through the National Institute of Health at the following website: “stemcells.nih.gov/info/scireport/appendixe.asp#eii,” and are commercially available through Chemicon (now a part of Millipore, Temecula, Calif.) or Invitrogen (Carlsbad, Calif.). For example, neurons may be identified by expression of neuronal markers B-tubulin III (neuron marker, Millipore, Chemicon), Tuj1 (beta-III-tubulin); MAP-2 (microtubule associated protein 2, other MAP genes such as MAP-1 or -5 may also be used); anti-axonal growth clones; ChAT (choline acetyltransferase (motoneuron marker, Millipore, Chemicon); Olig2 (motorneuron marker, Millipore, Chemicon), Olig2 (Millipore, Chemicon), CgA (anti-chromagranin A); DARRP (dopamine and cAMP-regulated phosphoprotein); DAT (dopamine transporter); GAD (glutamic acid decarboxylase); GAP (growth associated protein); anti-HuC protein; anti-HuD protein; alpha-internexin; NeuN (neuron-specific nuclear protein); NF (neurofilament); NGF (nerve growth factor); gamma-NSE (neuron specific enolase); peripherin; PH8; PGP (protein gene product); SERT (serotonin transporter); synapsin; Tau (neurofibrillary tangle protein);anti-Thy-1; TRK (tyrosine kinase receptor); TRH (tryptophan hydroxylase); anti-TUC protein; TH (tyrosine hydroxylase); VRL (vanilloid receptor like protein); VGAT (vesicular GABA transporter), VGLUT (vesicular glutamate transporter).

Cranial neural crest stem cell (“CraNCSC”) are a multipotent cell type that can generate a wide variety of cell types, including cranial mesenchymal cells, peripheral neurons, skeleton, glia, melanocytes and smooth muscle. Thus, the cells are believed to have critical roles in organogenesis. The cells can be identified by a series of markers. Chung et al. (2009) has isolated proposed CraNCSC having a marker profile of CD44⁺, Sca-1⁺, CD24⁺, Thy-1⁺, c-Kit⁻ and CD133⁻. Applicants' CraNCSC isolated from murine embryo are identified by the marker profile: neural crest markers (AP-2α, Twist1, and Snail1), (while mass cultured neural crest express Snail1, clonal culture express Snail2 instead of Snail1.) Motohashi et al. (2007) Stem Cells 25(2):402-10 isolated cells that were not shown to have self-renewing ability of mulipotent clones. It is critical to show multipotency from a single cell that is also capable of self-renewing in order to call them stem cells. In the literature, it has been suggested that these bHLH family proteins have redundant function. Also these cells express the neural crest stem cell marker (nestin). The majority of cells are positive for CD44, and Sca-1 which are cell surface antigens for stem cells and EGFP a transgenic reporter protein expressed in cell cytoplasm. In this system of Wnt1-Cre;R26R-EGFP, it serve as neural crest-lineage tracer. A marker expression analysis for the CraNCSC clone is provided in Table 1 and Table 2. Minimal positive marker expression or CraNCSC isolated from mammalian embryonic tissue, ES cell and iPSC is: CD164, CD151, CD109, CD34, CD55, CD47, CD82, CD320, CD248, CD302, CD200, CD38, CD276, CD68, CD14, CD93, CD274, CD97, CD33, Ly96, Ly6e. Additional markers are identified herein.

The CraNCSC can also be identified by its multipotency, e.g., the capacity to differentiate into at least one cell type selected from the group of an osteoblast, a chondrocyte, a smooth muscle cell a glial cell, a neuronal cell or an adipocyte using the appropriate culture conditions and medium.

The term “CraNCSC treatable disease or condition” is an inclusive term encompassing acute and chronic conditions, disorders or diseases of the tissue for which CraNCSC differentiates, e.g., treatment of a critical size defect in cranial skeletal bone, skeletal tissue or muscle including joints and neural defects. It can be a condition that requires treatment of healing, reinforcement, strengthening or the replacement of bone or cartilage. It can include a condition that autologous transplantation and synthetic material implantation of bone or cartilage will improve or ameliorate the symptoms of It can be an oral or condition requiring maxillofacial surgery as well as orthopedic surgery. A CraNCSC treatable disease or condition may be age-related, or it may result from injury or trauma, or it may be related to a specific disease or disorder. Acute conditions include, but are not limited to, conditions associated with neuronal cell death or compromise including cerebrovascular insufficiency, focal or diffuse brain trauma, diffuse brain damage, spinal cord injury or peripheral nerve trauma, e.g., resulting from physical or chemical burns, deep cuts or limb severance. The term also includes chronic conditions, e.g., chronic epileptic conditions associated with neurodegeneration, motor neuron diseases including amyotrophic lateral sclerosis, degenerative ataxias, cortical basal degeneration, ALS-Parkinson's-Dementia complex of Guam, subacute sclerosing panencephalitis, Huntington's disease, Parkinson's disease, synucleinopathies (including multiple system atrophy), primary progressive aphasia, striatonigral degeneration, Machado-Joseph disease/spinocerebellar ataxia type 3 and olivopontocerebellar degenerations, Gilles De La Tourette's disease, bulbar and pseudobulbar palsy, spinal and spinobulbar muscular atrophy (Kennedy's disease), primary lateral sclerosis, familial spastic paraplegia, Werdnig-Hoffmann disease, Kugelberg-Welander disease, Tay-Sach's disease, Sandhoff disease, familial spastic disease, Wohlfart-Kugelberg-Welander disease, spastic paraparesis, progressive multifocal leukoencephalopathy, familial dysautonomia (Riley-Day syndrome), and prion diseases (including, but not limited to Creutzfeldt-Jakob, Gerstmann-Straussler-Scheinker disease, Kuru and fatal familial insomnia), demyelination diseases and disorders including multiple sclerosis and hereditary diseases such as leukodystrophies. Additional CraNCSC treatable diseases or conditions include, for example, Apert syndrome, Boston-type craniosynostosis, Branchio-oto-renal syndrome, Cardio-facio-cutaneous syndrome, Cleft lip and palate, Craniosynostosis, DiGerge syndrome, Ewing sarcoma, Ganglioneuroma, Head and neck cancer including HNSCC (head and neck squamous cell carcinomas), Hirschsprung disease, LEOPARD syndrome, Melanoma, Neuroblastoma, Neurofibroma, Noonan syndrome, Oral-facial-digital syndromes, Pfeiffer syndrome, Saethre-chotzen syndrome, Townes-Brocks syndrome, Treacher collins syndrome, Waardenburg syndrome, and Waardenburg-Shah syndrome.

The term treating (or treatment of) a disease, disorder or condition refers to ameliorating the effects of, or delaying, halting or reversing the progress of, or delaying or preventing the onset of, an CraNCSC treatable disease, disorder or condition as defined herein.

The term “effective amount” refers to a concentration or amount of a reagent or composition, such as a composition as described herein, cell population or other agent, that is effective for producing an intended result, including cell growth and/or differentiation in vitro or in vivo, or for the treatment of a CraNCSC treatable disease, disorder or condition such as a critical size defect in cranial skeletal bone. It will be appreciated that the number of cells to be administered will vary depending on the specifics of the disorder to be treated, including but not limited to size or total volume/surface area to be treated, as well as proximity of the site of administration to the location of the region to be treated, among other factors familiar to the medicinal biologist and/or treating physician.

The terms effective period (or time) and effective conditions refer to a period of time or other controllable conditions (e.g., temperature, humidity for in vitro methods), necessary or preferred for an agent or composition to achieve its intended result, e.g., the differentiation of cells to a pre-determined cell type.

The term patient or subject refers to animals, including mammals, such as murine, canine, equine, bovine, simian or humans, who are treated with the pharmaceutical compositions or in accordance with the methods described herein.

The term pharmaceutically acceptable carrier (or medium), which may be used interchangeably with the term biologically compatible carrier or medium, refers to reagents, cells, compounds, materials, compositions, and/or dosage forms that are not only compatible with the cells and other agents to be administered therapeutically, but also are, within the scope of sound medical judgment, suitable for use in contact with the tissues of human beings and animals without excessive toxicity, irritation, allergic response, or other complication commensurate with a reasonable benefit/risk ratio. Pharmaceutically acceptable carriers suitable for use in the present invention include liquids, semi-solid (e.g., gels) and solid materials (e.g., cell scaffolds and matrices, tubes sheets and other such materials as known in the art and described in greater detail herein). These semi-solid and solid materials may be designed to resist degradation within the body (non-biodegradable) or they may be designed to degrade within the body (biodegradable, bioerodable). A biodegradable material may further be bioresorbable or bioabsorbable, i.e., it may be dissolved and absorbed into bodily fluids (water-soluble implants are one example), or degraded and ultimately eliminated from the body, either by conversion into other materials or breakdown and elimination through natural pathways.

The terms autologous transfer, autologous transplantation, autograft and the like refer to treatments wherein the cell donor is also the recipient of the cell replacement therapy. The terms allogeneic transfer, allogeneic transplantation, allograft and the like refer to treatments wherein the cell donor is of the same species as the recipient of the cell replacement therapy, but is not the same individual. A cell transfer in which the donor's cells and have been histocompatibly matched with a recipient is sometimes referred to as a syngeneic transfer. The terms xenogeneic transfer, xenogeneic transplantation, xenograft and the like refer to treatments wherein the cell donor is of a different species than the recipient of the cell replacement therapy.

A “control” is an alternative subject or sample used in an experiment for comparison purpose. A control can be “positive” or “negative”. For example, where the purpose of the experiment is to determine a correlation of an altered expression level of a gene with a particular phenotype, it is generally preferable to use a positive control (a sample from a subject, carrying such alteration and exhibiting the desired phenotype), and a negative control (a subject or a sample from a subject lacking the altered expression or phenotype). Additionally, when the purpose of the experiment is to determine if an agent effects the differentiation of a stem cell, it is preferable to use a positive control (a sample with an aspect that is known to affect differentiation) and a negative control (an agent known to not have an affect or a sample with no agent added).

### DETAILED EMBODIMENTS OF THE INVENTION

In one aspect, this invention provides an isolated self-renewable cranial neural crest stem cell (CraNCSC). The isolated self-renewable cranial stem cell can be isolated from any source, examples of which include without limitation, any animal (alive or dead) so long as the tissue containing the cranial neural stem cell is viable. Suitable tissue sources of CraNCSCs include, but are not limited to embryos, embryonic stem cells, such as the non-fetal and adult tissues as well as pluripotent stem cells including embryonic stem cells, parthenogenetic cells and iPSC. Thus, the isolated CraNCSC can be animal, e.g., mammalian such as equine, canine, porcine, bovine, murine, simian, and human.

The CraNCSC is isolated from the tissue source by any means that allows for isolation of a single cell by use of an identifying marker, e.g., FACS analysis. Details of this procedure are provided in Example 1, infra. Embryonic tissue, embryonic stem cells and adult tissues as well as pluripotent stem cells can be analyzed using a WNT1-CRE; R26R-EGFR reporter as described in (Chai et al. (2000); Jiang et al. (2000)) or other cell surface markers and intracellular markers as shown in Table 1, below. The isolated cells are then cultured in a combination of Matrigel or fibronectin-coated dishes as described in Xu, et al. (2001); Rovasio et al. (1983), with medium conditioned with STO feeder cells (SIM, 6-thioguanine resistant, ouabain resistant, as described in Kubota et al. (2004)). The cells are then cultured in STO-conditioned medium supplemented with about a range of bFGF as described herein, e.g. 25 ng/ml bFGF and a range of LIF as described herein, e.g., 1000 U LIF. Typically, mass culture need to be passaged about every 3 days. For clonal cultures, the cells were passaged after three weeks from the initial seeding.

In one aspect, the isolated CraCNSC are isolated using FACS analysis and the stem cell markers. Minimal positive marker expression or CraNCSC isolated from human embryonic tissue, ES cell and iPSC is: CD164, CD151, CD109, CD34, CD55, CD47, CD82, CD320, CD248, CD302, CD200, CD38, CD276, CD68, CD14, CD93, CD274, CD97, CD33, Ly96, Ly6e. Other confirmatory antigens are identified in Table 1 and described below and within Table 2.

TABLE 1

| Antigen or Marker | GenBank Accession No. |
|---|---|
| CD81 | NM_133655 |
| CD164 | NM_016898 |
| CD151 | NM_009842 |
| CD24a | NM_009846 |
| CD9 | NM_007657 |
| Ly6e | NM_008529 |
| CD109 | NM_153098 |
| CD44 | NM_009851 |
| CD2ap | NM_009847 |
| CD55 | NM_010016 |
| CD34 | NM_001111059 |
| CD99l2 | NM_138309 |
| Ly96 | NM_016923 |
| CD82 | NM_007656 |
| CD2bp2 | NM_027353 |
| CD47 | NM_010581 |
| CD320 | NM_019421 |
| CD3eap | NM_145822 |
| CD248 | NM_054042 |
| CD59a | NM_001111060 |
| CD38 | NM_007646 |
| CD200 | NM_010818 |
| CD302 | NM_025422 |
| Ly6a (Sca1) | NM_010738 |
| CD276 | NM_133983 |
| CD68 | NM_009853 |
| CD14 | NM_009841 |
| CD93 | NM_010740 |
| Thy1 | NM_009382 |
| CD274 | NM_021893 |
| CD97 | NM_011925 |
| CD33 | NM_001111058 |
| CD300lg | NM_027987 |
| CD5 | NM_007650 |
| CD46 | NM_010778 |
| CD177 | NM_026862 |
| CD3e | NM_007648 |
| CD1d1 | NM_007639 |
| CD37 | NM_007645 |
| CD1d2 | NM_007640 |
| CD163l1 | NM_172909 |
| CD79b | NM_008339 |
| CD2 | NM_013486 |
| CD8b1 | NM_009858 |
| CD300lf | NM_145634 |
| CD6 | NM_009852 |
| CD28 | NM_007642 |
| CD247 | NM_001113394 |
| CD300c | NM_199225 |
| CD209a | NM_133238 |
| CD40 | NM_170701 |
| CD8a | NM_001081110 |
| CD300lb | NM_199221 |
| CD300e | NM_172050 |
| CD7 | NM_009854 |
| CD74 | NM_001042605 |
| CD19 | NM_009844 |
| CD209c | NM_130903 |
| CD209e | NM_130905 |
| CD163 | NM_053094 |
| CD40lg | NM_011616 |
| CD72 | NM_001110320 |
| CD244 | NM_018729 |
| CD83 | NM_009856 |
| CD70 | NM_011617 |
| CD209d | NM_130904 |
| CD300a | NM_170758 |
| CD48 | NM_007649 |
| CD207 | NM_144943 |
| CD4 | NM_013488 |
| CD160 | NM_018767 |
| CD22 | NM_001043317 |
| CD180 | NM_008533 |
| CD84 | NM_013489 |
| CD200r2 | NM_206535 |
| CD53 | NM_007651 |
| CD3d | NM_013487 |
| CD200r4 | NM_207244 |
| CD5l | NM_009690 |
| CD96 | NM_032465 |
| CD226 | NM_178687 |
| CD79a | NM_007655 |
| CD200r3 | NM_001128132 |
| CD200r4 | NM_207244 |
| CD36 | NM_007643 |
| CD86 | NM_019388 |
| CD52 | NM_013706 |
| CD3g | NM_009850 |

TABLE 2

| Antigen or Marker | GenBank Accession No. |
|---|---|
| AP-2a | NM_011547 |
| Twist1 | NM_011658 |
| Snail2 | NM_011415 |
| Msx2 | NM_013601 |
| Dlx1 | NM_010053 |
| Dlx2 | NM_010054 |
| Pax3 | NM_008781 |
| Ets1 | NM_011808 |
| Foxc1 | NM_008592 |
| Crabp1 | NM_013496 |
| Cadherin6 | NM_007666 |
| Cnbp | NM_013493 |
| Eif4a2 | NM_001123038 |
| Ets2 | NM_011809 |
| Gli3 | NM_008130 |
| Myc | NM_010849 |
| Sox4 | NM_009238 |
| Sox9 | NM_011448 |
| Tcof1 | NM_011552 |
| Cdh11 | NM_009866 |
| Cdc4 is Fbxw7 | |
| Fbxw7 | NM_080428 |
| Fmr1 | NM_008031 |
| Fn1 | NM_010233 |
| Fxr1 | NM_001113188 |
| Fzd3 | NM_021458 |
| Fzd6 | NM_008056 |
| Fzd7 | NM_008057 |
| Gdnf | NM_010275 |
| Id2 | NM_010496 |
| Meis1 | NM_010789 |
| Myo10 | NM_019472 |
| Notch1 | NM_008714 |
| Nrp1 | NM_008737 |
| Nrp2 | NM_001077403 |
| Rhob | NM_007483 |
| Robo1 | NM_019413 |
| Sulf2 | NM_028072 |
| Zic2 | NM_009574 |
| Adh5 | NM_007410 |
| Akap1 | NM_009648 |
| Aldh9a1 | NM_019993 |
| Ankrd17 | NM_030886 |
| Atp1a1 | NM_144900 |
| Basp1 | NM_027395 |
| Bid | NM_007544 |
| Cachd1 | NM_198037 |
| Ccar1 | NM_026201 |
| Ccnb2 | NM_007630 |
| Ciapin1 | NM_134141 |
| Col4a5 | NM_007736 |
| Ctcf | NM_181322 |
| Ctnna1 | NM_009818 |
| Ctsb | NM_007798 |
| Ddx23 | NM_001080981 |
| Elk3 | NM_013508 |
| Ewsr1 | NM_007968 |
| G3bp1 | NM_013716 |
| Gart | NM_010256 |
| Glg1 | NM_009149 |
| Gnl2 | NM_145552 |
| Gtf2e1 | NM_028812 |
| Gstcd | NM_026231 |
| H3f3b | NM_008211 |
| Heph | NM_010417 |
| Hk2 | NM_013820 |
| Hnrnpa2b1 | NM_016806 |
| Hnrnpm | NM_029804 |
| Hp1bp3 | NM_001122897 |
| Ilf2 | NM_026374 |
| Ilf3 | NM_010561 |
| Ipo9 | NM_153774 |
| Ktn1 | NM_008477 |
| Lmnb2 | NM_010722 |
| Macf1 | NM_009600 |
| Mcm2 | NM_008564 |
| Mcm5 | NM_008566 |
| Mkrn1 | NM_018810 |
| Msh6 | NM_010830 |
| Nes | NM_016701 |
| Nf2 | NM_010898 |
| Nsun5 | NM_145414 |
| Psmd3 | NM_009439 |
| Ptprf | NM_011213 |
| Pxn | NM_011223 |
| Rbm4 | NM_009032 |
| Rcc2 | NM_173867 |
| Rnh1 | NM_145135 |
| Sec14l1 | NM_028777 |
| Srebf2 | NM_033218 |
| Srf | NM_020493 |
| Taldo1 | NM_011528 |
| Tcf20 | NM_001114140 |
| Thoc5 | NM_172438 |
| Tnrc18 | NM_001122730 |
| Tpd52l2 | NM_025482 |
| Tpm3 | NM_022314 |
| Trio | NM_001081302 |
| Vav2 | NM_009500 |
| Whsc1l1 | NM_001081269 |

TABLE 3

| Antigen or Marker | GenBank Accession No. |
|---|---|
| Ret | NM_001080780 |
| CD9 | NM_007657 |
| Sox10 | NM_011437 |
| Gfra1 | NM_010279 |
| Gas7 | NM_001109657 |
| Ednrb | NM_007904 |
| Ccnd1 | NM_007631 |
| Hsp90 | NM_008302 |
| Cox2 | NM_011198 |
| Vim | NM_011701 |
| Hif1a | NM_010431 |
| Myc | NM_010849 |
| Mcl1 | NM_008562 |
| Birc5 | NM_001012273 |
| Vegfa | NM_001025250 |
| Vegfb | NM_011697 |
| Vegfc | NM_009506 |
| Twist1 | NM_011658 |
| Cxcl12 | NM_001012477 |
| Il-11 | NM_008350 |
| Icam1 | NM_010493 |
| Fgf2 | NM_008006 |

The isolated cell expresses neural crest markers (AP-2α, Twist1, and Snail1), and a neural crest stem cell marker (nestin). In addition, flow cytometory analysis showed that more than 97% of these cells are positive for CD44, and Sca-1.

In addition to the markers, the isolated cell is identifiable by its multipotency, e.g., it is capable of differentiation into at least one cell type selected from the group of an osteoblast, a chondrocyte, a smooth muscle cell a glial cell, a neuronal cell or an adipocyte using the appropriate culture conditions and medium. Confirmation of the differentiation state of the cells can be performed by identification of cell type specific markers as known to those of skill in the art. In one aspect, the isolated cranial neural crest stem cell is capable of differentiation into at least two of the cell types. In another aspect, the isolated cranial neural crest stem cell is capable of differentiation into at least two, or alternatively at three, or alternatively at least four, or alternatively at least five, or alternatively at least six of the cell types.

In a further aspect, the isolated cranial neural crest stem cell expresses one or more marker of the group CD44, Sca-1, nestin, AP-2α, Twistl, Snaill or EGFP. In a further aspect, the cell is a murine CraNCSC and expresses these markers. When the CraNCSC is a clonal cell, the clone expresses Snail2 instead of Snail1. In a yet further aspect, the cells express one or more of the markers identified in Table 1 or Table 2.

In a further aspect, the isolated CraNCSC expresses at least CD164, CD151, CD109, CD34, CD55, CD47, CD82, CD320, CD248, CD302, CD200, CD38, CD276, CD68, CD14, CD93, CD274, CD97, CD33, Ly96, Ly6e. In a further aspect, the cells are isolated from a human embryo, human embryonic stem cells, a human non-fetal tissue or human adult tissue. In a yet further aspect, the cells express one or more of the markers identified in Table 1 or Table 2.

The isolated cranial neural crest stem cell can be further identified by the ability to be passaged for at least about 10 times, or alternatively for at least about 30 times, or alternatively, at least about 100 times, or alternatively for at least about 1 month alternatively for at least about 3 months, or alternatively for at least about 6 month, when passaged on Matrigel or fibronectin coated plates in STO-conditioned medium supplemented with bFGF and LIF.

In a further aspect, this invention provides isolated clonal population of the isolated cranial neural crest stem cell as described above. The clonal population contains majorities of the characteristics of the isolated cell as identified above. As noted above, the clonal cells and clonal populations express one or more of markers CD44, Sca-1, nestin, AP-2α, Twist1, Snail2 or EGFP. In one aspect the cells express at least two, or alternatively at least three, or alternatively at least four, or alternatively at least five, or alternatively at least six, or alternatively all seven markers.

This invention also provides an isolated cranial neural crest stem cell wherein the isolated cranial neural crest stem cell expresses one or more marker of the group CD44, Sca-1, nestin, AP-2α, Twist1, Snail1, Snail2, CD93 or EGFP. In one aspect two markers are present, or alternatively, three, or alternatively four, or alternatively five, and increasing up to the presence of all markers.

The invention also provides an isolated cranial neural crest stem cell, wherein the isolated cranial neural crest stem cell further expresses the markers identified above with one or more marker of the group AP-2α, Twist1, Snail2, Msx2, Dlx1, Dlx2, Pax3, Ets1, Foxc1, Crabp1, and Cadherin6. In one aspect two markers are present, or alternatively, three, or alternatively four, or alternatively five, and increasing up to the presence of all markers.

Also provided is an isolated cranial neural crest stem, wherein the isolated cranial neural crest stem cell expresses as identified abve and yet further expresses one or more marker of the group D7-1; Cnbp, Eif4a2, Ets2, Gli3, Myc, Sox4, Sox9, Tcof1, Cdh11, Cdc4, Fbxw7, Fmr1, Fn1, Fxr1, Fzd3, Fzd6, Fzd7, Gdnf, Id2, Meis1, Myo10, Notch1, Nrp1, Nrp2, Rhob, Robo1, Sulf2, and Zic2. In one aspect two markers are present, or alternatively, three, or alternatively four, or alternatively five, and increasing up to the presence of all markers.

Yet further provided is an isolated cranial neural crest stem cell as described above, wherein the isolated cranial neural crest stem cell further expresses all of the markers.

In a further aspect, the isolated cranial neural crest stem cell expresses Sca-1 and at least one or more marker of the group CD44, nestin, AP-2α, Twist1, Snail1, Snail2, CD93 or EGFP. In one aspect two markers are present, or alternatively, three, or alternatively four, or alternatively five, and increasing up to the presence of all markers.

In another aspect, the isolated cranial neural crest stem cell expresses Sca-1 and CD93 at least one or more marker of the group CD44, nestin, AP-2α, Twist1, Snail1, Snail2, or EGFP. In one aspect two markers are present, or alternatively, three, or alternatively four, or alternatively five, and increasing up to the presence of all markers.

In yet another aspect, the isolated cranial neural crest stem cell is as described above and yet further expresses one or more marker of the group Gfra 1, CD81, CD9, CD34, CD47, CD38, CD200r, CD276, CD14, CD93 (AA4.1), CD274 or CD205. In one aspect two markers are present, or alternatively, three, or alternatively four, or alternatively five, and increasing up to the presence of all markers.

This invention also provides an isolated cranial neural crest stem cell as described above, wherein the isolated cranial neural crest stem cell further expresses one or more marker of the group LIFR, gp130, JAK1, JAK2, STAT1, STAT3, or STATS. In one aspect two markers are present, or alternatively, three, or alternatively four, or alternatively five, and increasing up to the presence of all markers.

This invention also provides an isolated cranial neural crest stem cell as described above, wherein the isolated cranial neural crest stem cell further expresses one or more marker of the group Ccnd1, Hsp90, Cox2, Vim, Hif1α, Myc, Mcl1, Birc5, Vegf, Twist1, Cxcl12, Il-11, Icam1, or Fgf2. In one aspect two markers are present, or alternatively, three, or alternatively four, or alternatively five, and increasing up to the presence of all markers.

In a further aspect, any of the isolated cells as described above do not expresses or only expresses at a low level one or more marker of the group Ret, Sox10, Gas7 or Ednrb. In one aspect two markers are not present, or alternatively, not three, or alternatively not four.

This invention also provides an isolated cranial neural crest stem cell as described above which further does not expresses or only expresses at a low level one or more marker of the group Sox17, Afp, and Pdx1, Mesp1, Mesp2, T, Gata4, Gsc, Nodal or a terminal differentiation marker for osteogenic, chondrogenic, smooth muscle, myogenic, neuronal, or Schwann cell. In one aspect two markers are absent, or alternatively, three are absent, or alternatively four are absent, or alternatively five are absent, and increasing up to the absence of all markers.

This invention also provides an isolated CraNCSC as described above or an isolated population of same further comprising an exogenous agent, e.g., a small molecule, detectable label (e.g., a label for use in FACs analysis), antibody or a non-naturally occurring nucleic acid, e.g. a therapeutic nucleic acid. Thus, these compositions are useful in the therapeutic and diagnostic methods as described herein as well as the screens for new therapeutic agents.

This invention also provides methods for isolating a CraNCSC and/or a method for preparing a substantially homogeneous population of isolated neural crest stem cells or populations as described. To isolate the CraNCSC, the method requires contacting a source cell, population or tissue likely to contain the CraNCSC with a detectably labeled antibody or other ligand that is specific for one or more identifying marker as identified above. After sufficient time and under appropriate conditions to allow the ligand to bind the marker to form a ligand-marker complex. The cells having the ligand-marker complex are then separated by any appropriate means, e.g., by FACs, from those that do not have a ligand-marker complex, thereby preparing an isolated CraNCSC.

In a further aspect, this invention provides a method for preparing a clonal population, a mass culture and/or differentiating an isolated neural crest stem cell as described above or the population as described above by contacting the cell or population with an effective amount of a clonal expansion medium or differentiation medium as described infra and culturing of the cells under the appropriate conditions to obtain any of a clonal population or a mass culture or yet further differentiation into a selected lineage. In one aspect, the method prepares an expanded substantially homogenous population of CraNCSCs, or alternatively glial cells, or alternatively, osteoblasts, or alternatively neurons, or alternatively adipose cells, or alternatively or alternatively chondrocytes, or alternatively, smooth muscle cells. These populations are useful in the therapeutic and diagnostic methods as described herein as well as the screens for new therapeutic agents. The contacting may be performed in vitro or in vivo, depending on the intended use. For example, the isolated cell or population of cell can be implanted (autologous or allogeneic) into a subject and appropriate conditions can be locally administered to induce expansion and/or differentiation. Alternatively, the microenvironment of the cells will induce the appropriate differentiation of the cells into to cells and tissue. Yet further, agents can be administered to the subject to induce local expression of the agents that in turn, induce expansion and differentiation.

The isolated cells and/or populations of cells as described herein can be further combined with carrier, e.g., a pharmaceutically acceptable carrier for ease of administration.

This invention also provides a stem cell growth medium comprising, or alternatively consisting essentially of, or yet further consisting of, stem cell culture medium supplemented with fetal bovine serum (FBS), basic fibroblast growth factor (bFGF) and optionally leukemia inhibitory factor (LIF). In a further aspect, the growth medium further comprising, or alternatively consisting essentially of, or yet further consisting of, one or more of Dulbecco's modified Eagle's medium (DMEM), about 0.1 mM MEM nonessential amino acids, about 0.1 mM sodium pyruvate, abou 55 μM β-mercaptoethanol, about 100 units/ml penicillin, about 100 units/ml streptomycin or about 2 mM L-glutamine. In a yet further aspect, the growth medium is conditioned by STO feeder cells. In a yet further aspect, medium is conditioned by STO feeder cells for at least about 24 hours.

In another aspect, the bFGF is present in a concentration from about 15 ng/ml to about 35 ng/ml, or alternatively from about 20 ng/ml to about 30 ng/ml, or alternatively from about 22 ng/ml to about 28 ng/ml, or alternatively about 25ng/ml.

In a further aspect, the medium is supplemented with FBS which is presented at a concentration of about 10% to 20%, or alternatively about 13% to about 17%, or yet further about 15%.

In yet further aspect, the LIF is presented at a concentration from about 700 U to about 1300 U, or alternatively from about 800 U to about 1200 U, or alternatively from 900 U to about 1100 U, or alternatively from about 1000 U.

In a yet further aspect, at least one of the above conditions, or alternatively at least two, or alternatively all three are present in the same medium.

The medium as described herein is useful to obtain an isolated clonal population of CraNCSC as described above. Thus in one embodiment, this invention provides a method of culturing a cranial neural crest stem cell comprising, or alternatively consisting essentially of, or yet further consisting of contacting an isolated CraNCSC as described above with the growth medium as described above under conditions that favor clonal expansion of the cell. In a further aspect, the conditions include plating on Matrigel or fibronectin coated plates or by the hanging drop method as described in Xu, et al. (2001); Rovasio et al. (1983); Wobus et al. (2002).

This invention also provides a kit for isolating, clonally expanding and/or differentiating a cranial neural crest stem cell as described above comprising, or alternatively consisting essentially of, or yet further consisting of, an effective amount of ligands and labels to isolate the cells and instructions for use. The kit may further comprise, or alternatively consist essentially of, or yet further consist of, Applicants' growth medium, and instructions for use of the growth medium. In a yet further aspect, the kit comprises, or alternatively consists essentially of, or yet further consists of, Matrigel and/or fibronectin coated plates and instructions for use.

The cell compositions as described herein are useful therapeutically and diagnostically. It should be noted that such knowledge of pathophysiology will lead to development of novel way of diagnosis or cure for those pathogenic conditions. In one aspect, this invention provides a method for ameliorating the symptoms of spinal cord injury or a CraNCSC treatable disease, disorder or condition as is apparent to those of skill in the art, in a subject in need thereof, comprising, alternatively consisting essentially of, or yet further consisting of administering to the subject an effective amount of the isolated cranial neural crest stem cell as described above or the population as describe herein thereby treating the CraNCSC treatable disease, disorder or condition. Methods of administering cell populations are well known in the art and will depend on the treatment and individual. One or more administrations may be necessary. The cells may be autologous, allogeneic syngeneic or xenogeneic to the subject being treated. The subjects can be mammalian, e.g., bovine, canine, equine or a human patient.

This invention also provides the use of the isolated cranial neural crest stem cell or the population of as described herein in the manufacture of a medicament. In one aspect, the medicament is to treat a CraNCSC treatable disease, disorder or condition.

Various diagnostic and high throughput screens are also disclosed. A method for identifying an agent that modulates the growth or differentiation of the isolated cranial neural crest stem cell is further provided by this invention. The method comprises, or alternatively consisting essentially of, or yet further consisting of, using the isolated cell or the population of cells and contacting the cell or the population with the agent wherein a change of growth or differentiation of the cell or population indicates that the agent modulates the growth or differentiation of the cell or the population. One can screen for agents that inhibit the growth or promote growth and/or differentiation of the cell or cell population.

Additional diagnostic uses include use as a tool for the research of neurocristopathy. The neurocristopathy is disease resulted from anomaly of the neural crest. That includes defects of neural crest development and tumors of neural crest descendants.

It should be noted that such knowledge of pathophysiology will lead to development of novel way of diagnosis or cure for those pathogenic conditions. Following are examples of diseases: Apert syndrome, Boston-type craniosynostosis, Branchio-oto-renal syndrome, Cardio-facio-cutaneous syndrome, Cleft lip and palate, Craniosynostosis, DiGerge syndrome, Ewing sarcoma, Ganglioneuroma, Head and neck cancer including HNSCC (head and neck squamous cell carcinomas), Hirschsprung disease, LEOPARD syndrome, Melanoma, Neuroblastoma, Neurofibroma, Noonan syndrome, Oral-facial-digital syndromes, Pfeiffer syndrome, Saethre-chotzen syndrome, Townes-Brocks syndrome, Treacher collins syndrome, Waardenburg syndrome, and Waardenburg-Shah syndrome.

This disclosure also provides methods to screen for cancer stem cell markers. Some of these markers are cell surface antigens that can be used for cancer treatment. Others screens provided herein include the analysis of molecular and cellular mechanisms of neural crest development defects and tumorigenesis.

One can identify putative cancer stem cells and their markers by utilizing database screening to identify stem cell markers. One compares different database sets of gene expression using the marker profile provided herein against the other gene expression profile. Using comparative screening, one of skill in the art can identify genes that correlate to 1) poor prognosis or aggressiveness of human cancer as well as 2) proliferation or stemness of craNCSC. Genes that belongs to both categories likely serve as cancer stem cell markers.

One can also use culture based screening. Cells are isolated from subjects such as humans and culture in the presence of craNCSC culture media and conditions. Cells that exhibit sustainable proliferation likely contain cancer stem cells. These cells are isolated from culture and used to perform serial cell implantation against different hosts (e.g., mice) to determine if they are capable of reproducing the original cancer. Those which do likely contain cancer stem cells. These cells are then subjected to whole genome expression profiling to determine markers of the putative cancer stem cell.

The present technology is further understood by reference to the following examples. The present technology is not limited in scope by the examples, which are intended as illustrations of aspects of the present technology. Any methods that are functionally equivalent are within the scope of the present technology. Various modifications of the present technology in addition to those described herein will become apparent to those skilled in the art from the foregoing description and accompanying figures. Such modifications fall within the scope of the appended claims.

### EXAMPLES

### Example 1

### Sustainable Stem-Like Potency of Mass Cultured Cranial Neural Crest

To date, only bipotent and unipotent progenitors have been reported to have self-renewing ability in the cranial neural crest population. This may be because culture conditions used in previous studies were not optimal. Therefore, it is surprising and unexpected that the new conditions discovered in the current invention support long-term growth of mouse cranial neural crest.

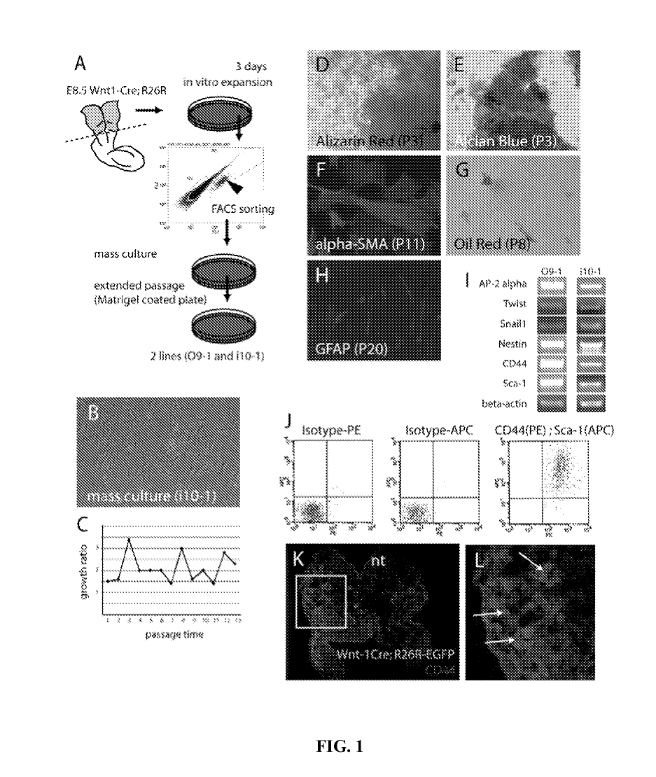

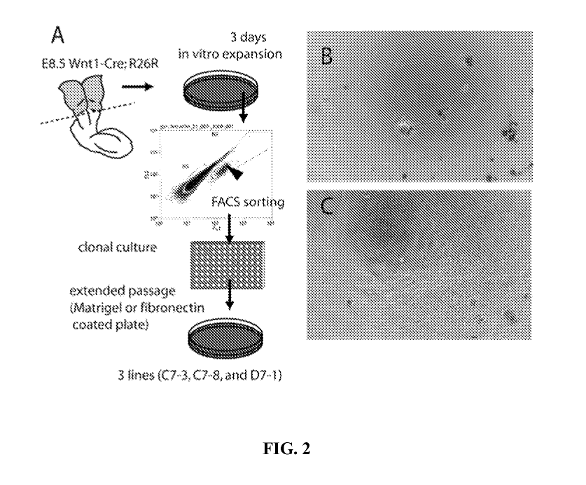

Cranial neural crest were isolated from E8.5 mouse embryos by means of Fluorescence Activated Cell Sorting (FACS) (FIG. 1). The cells were labeled with a Wnt1-Cre;R26R-EGFP reporter (Chai et al. (2000); Jiang et al. (2000)). Several different cell culture conditions were tested. By using a combination of Matrigel-coated culture dishes and medium conditioned medium by STO feeder cells and supplemented with bFGF (basic fibroblast growth factor) and LIF (leukemia inhibitory factor), sustainable growth of mouse cranial neural crest was obtained. Two independent mass culture lines were isolated. One (O9-1) has been passaged over 30 times for more than 3 months and continues to maintain its osteogenic potential. Intriguingly, these cells are also capable of differentiating into several different cell-types, including chondrocytes, smooth muscle cells, neurons, and glial cells. A second line exhibits similar characteristics. By performing RT-PCR analysis at different passages, it is found that both of these cell lines continually express neural crest markers (AP-2α, Twist1, and Snail1), and a neural crest stem cell marker (nestin). In addition, flow cytometory analysis showed that more than 97% of these cells are positive for EGFP (neural crest), CD44, and Sca-1 which are cell surface antigens for stem cells. Thus, findings of sustainable osteogenic potential, multipotency and marker gene expression suggest that these mass cultured cells are a stem-like population of cranial neural crest. Moreover, these data show that the culture conditions of the current invention are capable of supporting the self-renewal of these cells.

Basal medium was prepared as follows. Dulbecco's modified Eagle's medium (DMEM), 15% Fetal bovine serum (FBS), 0.1 mM MEM nonessential amino acids, 1mM sodium pyruvate, 55 μM β-mercaptoethanol, 100 units/ml penicillin, 100 units/ml streptomycin, and 2 mM L-glutamine was conditioned by STO feeder cells for an overnight. Medium was filtered (0.22 μm pore size) and supplemented with 25 ng/m1 bFGF and 1000 U LIF.

### Example 2

### Clonal Culture of Cranial Neural Crest Cells

To demonstrate that the cranial neural cells isolated are multipotent and have the ability to self-renew, it is crucial to develop cultivated, clonal lines. Using culture conditions similar to those used for mass culture of cranial neural crest, three independent clones of cranial neural crest were established (FIG. 2). First, with Wnt1-Cre;R26R-EGFP as in Example 1, neural crest cells from E8.5 mouse embryos were isolated. Single cells were then seeded on 96 well plates with growth-inhibited STO feeder cells at low cell density (10³ cells/cm²). Then, these cells were cultured in STO-conditioned medium supplemented with bFGF and LIF. After 3 weeks, the cells were passaged. For passaging, cells were detached from culture plates with 0.05% trypsin in 0.5 mM EDTA. For following passages, the cells were seeded to new wells coated with Matrigel or fibronectin.

### Example 3

### Differentiation Potential of Cranial Neural Crest Clones

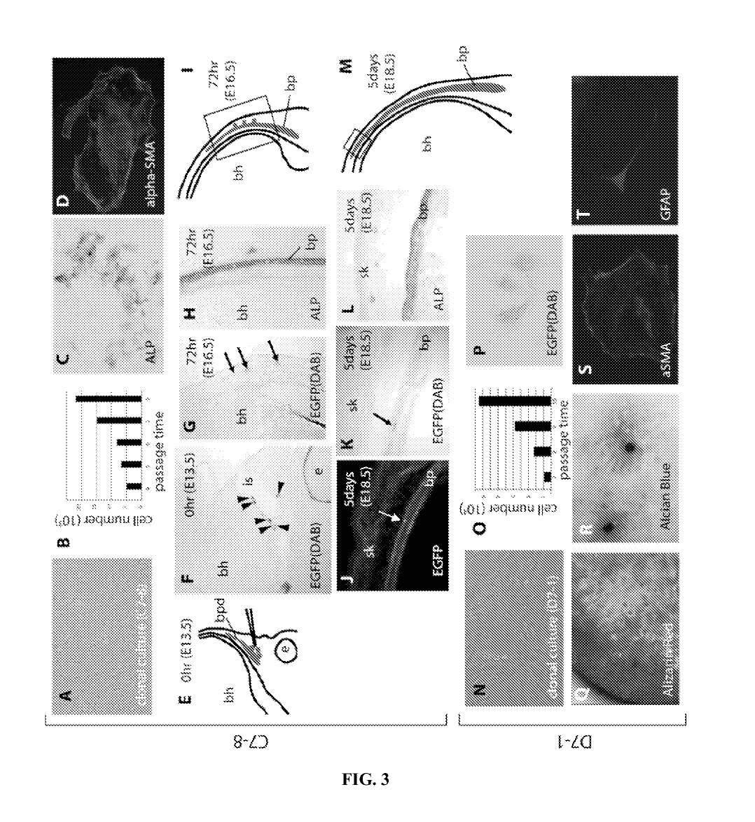

The ability of the newly-isolated cranial neural crest clones to differentiate into different cells types was tested (FIG. 3). One of these clones (#C7-8) expressed CD44, Sca-1, and nestin and had osteogenic potential. C7-8 cells treated with osteogenic medium for 3 weeks expressed the early osteoblast differentiation marker, ALP (alkaline phosphatase), (FIG. 3). Moreover, when these cells were implanted into the primordium of the frontal bone at E13.5, they behaved like normal calvarial bone precursors—migrating apically together with host osteoblast precursors, eventually becoming incorporated into calvarial bone at E18.5. To the Applicants' knowledge, this was the first clone of cranial neural crest that exhibited osteogenic potential in vivo, in the developing embryo. This clone also differentiated into smooth muscle when cultured in TGF-β supplemented medium (FIG. 3). Clone #D7-1 was also demonstrated to be multipotent, able to differentiate into osteoblasts, chondrocyte, and glia cells in vitro (FIG. 3). Media and procedures for inducing there differentiations are described as follows.

### A. Osteogenic Differentiation

### A-1. Short-Term Culture

Cells were trypsinized, neutralized and centrifuged. Cells were resuspended to the basal medium at high cell density (ranges from 770 to 1900 cells/μl) and cultured in 30 μl hanging-drop format. By 12 hrs, the early osteoblast differentiation maker ALP (alkaline phosphatase) expression became apparent. From 24 hrs through 27 hrs, the mineralized bone matrix stained by alizarin red became prominent.

### A-2. Lone-Term Culture-1

Twenty-four hours cultured hanging-drops were seeded to the cell culture dish and maintained in osteogniec differentiation medium (αMEM, 10% FBS, 100 units/ml penicillin, 100 μg/ml streptomycin, 0.104 dexamethasone, 10 mM β-glycerophosphate, 50 μg/ml ascorbic acid, with or without 100 ng/ml BMP2) for 2 to 3weeks. The medium was changed every 3 days. In the end of culture, cells were stained for alizarin red.

### A-3. Lone-Term Culture-2 (Micromass Culture)

Approximately 37000 cells were trypsinized, neutralized and centrifuged. By using 15 ml tube, cell pellet was cultured in 250 μl of osteogniec differentiation medium for 2 to 3 weeks. The medium was changed every 3 days. Cell aggregates were fixed by 4% PFA and cryosectioned. Tissues are positively stained by alizarin red.

### B. Chondrogenic Differentiation

Twenty-four hours cultured hanging-drops were seeded to the cell culture dish and maintained in chondrogenic differentiation medium (αMEM, 10% FBS, 10 ng/ml TGFβ-3, 50 μg/ml ascorbic acid) for 2 weeks. The medium was changed every 3 days. In the end of culture, cells were stained for alcian blue.

### C. Smooth Muscle Differentiation

Approximately 500 cells per cm² were seeded to the cell culture dish. They were cultured in smooth muscle differentiation medium (DMEM, 10% FBS, 100 units/ml penicillin, 100 μg/ml streptomycin) for 7 days. The medium was changed every 2 to 3 days. Smooth muscle differentiation was confirmed by examining αSMA expression.

Applicants discovered that this procedure can be modified by using cell culture plates pre-coated with 20 μg/ml fibronectin and that culture duration can be extended to 2 weeks. Additionally, initial seeding concentration can be increased to about 5000 cells per cm².

### D. Glial Differentiation

Cells were cultured in glial differentiation medium (DMEM, 1% FCS, 100 units/ml penicillin, 100 μg/ml streptomycin, 25 ng/ml bFGF) for 7 days. The medium was changed every 2 to 3 days. Glial differentiation was confirmed by examining GFAP expression.

Applicants also found that seeding the cells at lower cell density (1000 to 5000 cells/cm2) to Lab-Tek Chamber Slide (Nunc, product # 177399) improves differentiation into glial cells. The Lab-Tek slide is a 4 chambered slide culture system that is mounted on a glass microscope slide. Originally, the slide itself does not have any type of coating applied to its surface. However, when coated with 20 μg/ml fibronectin (diluted in DMEM) for one hour at room temperature and then dry. Applicants also found that shorter culture period (total 2 to 3 days) produces better results. Medium can be changed every day.

### Example 4

### To Further Investigate Putative Stem Cell Populations Isolated from the Murine Cranial Neural Crest

It had been thought that multipotent, stem-like cells of the cranial neural crest do not self-renew. Thus the identification of three passageable clones of cranial neural crest stem cells in the current invention was surprising and unexpected. Thus, Applicants contemplate that the cranial neural crest stem cell clones can be directed to other cell-lineages including adipocytes, and neurons.

Additional cranial neural crest stem cell clones can be established. Further, CD44, Sca-1, and other cell surface markers on Table 1 can be used as tools to sort undifferentiated stem-like cells from other cells to establish auxiliary clones more efficiently. It has been shown that these cell surface antigens are consistently expressed in mass cultured stem-like cells. In addition, CD44 is expressed in the majority of migratory cranial neural crest cells in E8.0 mouse embryos. Its expression declines significantly by E9.0. Accordingly, CD44 may identify a stem cell population in cranial neural crest and, together with Sca-1, may provide a marker with which to enrich a stem cell population. This strategy is also supported by a recent finding that both Sca-1 and CD44 are expressed in cultured neural crest stem-like cells from mandibular tissue (Chung et al. (2009)).

It can also be determined in the invention whether the self-renewal ability of cranial neural crest clones is a fundamental feature of their biology. That such clones have been established makes an argument ipso facto for the ability of these cells to self-renew.

It has been observed in the invention that mass-cultured stem-like cranial neural crest lines show a unique combination of marker gene expression (Twist, Snail1, Sca-1, and nestin) which was previously reported in SKPs (skin-derived precursors) (Fernandes et al. (2004) and COPs (corneal precursors) (Yoshida et al. (2006)), which are adult neural crest-derived stem cells. Intriguingly, the adult tissues in which they reside are cranial neural crest derivatives (the whisker follicle dermal papillae for SKPs and the cornea for COPs). It is also important to note that all of them share mesenchymal-lineage differentiation potential with bone marrow-derived mesenchymal stem cells (MSCs). Further, CD44, Sca-1, and Thy-1, well established representative markers of MSCs (da Silva Meirelles et al. (2006)), are present in cultured cranial neural crest cells (Chung et al. (2009)). Thus, it is predictable that there are links between stem cells originating from embryonic cranial neural crest and its adult descendants as well as adult bone marrow stroma. Raising the possibility that cranial neural crest cells may resemble ES cells is the finding that long-term cultured human trunk neural crest show more similarity to pluripotent ES cells than MSCs (Thomas et al. (2008)). These cells express pluripotent marker NANOG, POU5F1 and SOX2 as well as mixed spontaneous expression of αSMA, TUJ1, or GFAP.

To induce differentiation, cells are exposed to the following culture conditions: Osteogenic differentiation medium (αMEM, 10% FBS, 100 units/ml penicillin, 100 μg/ml streptomycin, 0.1 μM dexamethasone, 10 mM β-glycerophosphate, 50 μg/ml ascorbic acid, 100 ng/ml BMP2), chondrogenic differentiation medium (αMEM, 10% FBS, 10 ng/ml TGFβ-3, 50 μg/ml ascorbic acid), smooth muscle differentiation medium (αMEM, 10% FBS, 100 units/ml penicillin, 100 μg/ml streptomycin), adipogenic differentiation medium (αMEM, 10% FBS, 1 mM dexamethasone, 10 mg/ml insulin, 0.5 mM isobutyl-xanthine), neuronal differentiation medium (N2 medium supplemented with 20 ng/ml brain-derived neurotrophic factor (BDNF), 10 ng/ml nerve growth factor (NGF), 10 ng/ml glial cell line-derived neurotrophic factor (GDNF), 1 mM dibutyryl cAMP), glial differentiation medium (DMEM/F12, 1% FBS, 1×B27, 2 mM L-Glutamine, 100 units/ml penicillin, 100 μg/ml streptomycin, 50 ng/ml BMP2, 50 ng/mL LIF).

Marker analysis will determine whether the cells have undergone differentiation. Exemplary markers are as follows. For the osteogenic lineage, ALP expression and Alizarin red staining can be used; for the chondrogenic lineage, type-II collagen expression and Alcian blue staining; for the myofibroblastic lineage; α-SMA and SM22α expression; for the adipogenic lineage; PPARy expression and Oil-O-Red staining; for the neuronal lineage, neurofilament and Tuj1 expression; for the glial lineage, GFAP and S100b expression.

### Example 5

### To Determine Osteoprogenitors Derived from Cranial Neural Crest Stem Cells are Capable of Repairing Critical Size Defects in the Skull Vault

Accidental injury, diseases resulting in tissue degeneration, congenital disorders, and surgical treatment of tumors all can produce severe deficiencies in the craniofacial skeleton. Since critical size defects in the cranial bones by definition do not heal autonomously in adult humans, elaborate reconstructive surgeries are required to treat them. Cranial neural crest forms the majority of the craniofacial skeleton including the frontal, nasal, maxillary and mandibular bones. Thus, cranial neural crest stem cells may be an effective therapeutic biomaterial for craniofacial skeletal reconstruction. Animal model using the compositions and methods as described herein are useful to construct an animal model of craniofacial bone injury consisting of a critical-size defect in the skull vault of immunocompromised mice. The current invention shows that a clonally derived cranial neural crest cell line is capable of contributing to calvarial bone, in vivo (FIG. 3). The cultured cranial neural crest clones from this line and line D7-1 are implanted, as well as from a mass-cultured line that can also undergo osteogenic differentiation (FIG. 1). Applicants contemplate that osteogenic grafts derived from cranial neural crest stem cell will exhibit a shortened healing period, minimal resorption, maintenance of proper bone matrix density, proximity to the recipient site, revascularization, no tumorigenesis, and no additional abnormalities.

Cranial neural crest cells from both mass and clonal culture are used. In the case of mass cultured cells, the existing lines (O9-1 and i10-1) can be used as examples of such cell sources. In the case of clonally cultured cells, lines with osteogenic potential beginning with clone #C7-8 and D7-1 are preferred. Cells can be passaged up to at least passage 10. Adult MSCs are useful as a positive control. These cells have been shown to promote healing of critical size defects. A priori it is believed that neural crest stem cells are likely to be more effective than MSCs because much of the craniofacial skeleton is derived from neural crest rather than mesoderm—from which MSCs are derived.

To utilize the ability of neural crest stem cells to ameliorate craniofacial bone injuries, apatite coated-PLGA seeded with cultured cranial neural crest cells are grafted. Apatite coated-PLGA has been successfully used to repair critical size defects in calvarial bone with a combination of ADAS (adipose derived adult stromal) cells or bone marrow MSCs (Cowan et al. (2004)). Cranial neural crest cells are seeded at 8×10⁴ cells per cm² of scaffold and cultured in the basal medium. 24 hours after seeding, the attachment of cells to the scaffold are examined by DAPI staining Forty-eight hours after seeding, grafts are implanted to the host animals. Critical size defects in mouse skull bones (4 mm in diameter) are introduced without disrupting the dura matter (Cowan et al. (2004)). To avoid immunorejection, immunocompromised beige mice (NIH-bg-v/v-xid) are the preferred hosts. The graft disc will be implanted in the defect site of 10 weeks old male mice and the skin is sutured. Surgery is performed on 6 animals for each graft. Scaffold without seeded cells serves as a negative control. The outcome of experimental and control graft implantations is evaluated. This method utilizes cranial neural crest stem cells to ameliorate craniofacial bone injuries and other disease conditions.

### Example 6

### Whole Genome Analysis

Analyzing the whole genome transcriptome is an important research tool in understanding the status of gene regulatory networks in different cell-types, including stem cells. Applicants determined the whole genome gene expression profile of cranial neural crest stem cells by means of an Affymetrix mouse exon microarray analysis. Triplicated experiments were carried out for one cell line.

Equivalent level of the transcripts were detected in 16637 genes among these triplicates. Importantly, Applicants observed consistent expression of neural crest markers including AP-2α, Twist1, Snail2, Msx1, Msx2, Dlx1, Dlx2, Pax3, Ets-1, Foxc1, Crabp1, and Cadherin6. Stem cell marker CD44, Sca-1, CD24, and Nestin are expressed. Also, the expression of genes involved in JAK/STAT pathway (LIFR, JAK1, JAK2, and STAT3) and BMP signaling pathway (Bmpr1a, Bmpr2, Smad1, Smad3, and Smad5) are evident. This is particularly noteworthy because these are the pathways known to be involved in the molecular networks that promote pluripotency of mouse embryonic stem cells. Finally the results showed the transcriptional activation of genes that encode various cell surface antigens (see Table1.). A majority of these genes were not previously reported in the neural crest stem cell studies.

Thus, whole genome transcription can serve as a fundamental descriptor, providing a means of categorizing the neural crest clones relative to other stem-like cells, and also providing a first approximation view of the regulatory networks that may control their development.

### Example 7

### Unique CraNCSC Markers

A unique combination of cell surface antigen defines varieties of cell-types in diverse range of differentiation status. By means of antibodies against specific cell surface antigens and FACS sort or equivalent procedures, one can isolate and enrich stem cell populations from surrounding embryonic tissues or adult tissues, as well as differentiated pluripotent stem cell such as ES cell or iPS cells. Thus, identification of specific cell surface antigen is essential in order to purify and culture particular stem cells in undifferentiated status.

### Cranial Neural Crest Clone D7-1 Culture

Monolayer and hanging-drop culture of craNCSC clone D7-1 was performed as described above.

### Clonal Culture of Cranial Neural Crest Based on CD44 and Sca-1 Selection

Cranial neural crest cells marked with Wnt1-Cre;R26R-EGFP were obtained from

E8.5 mouse neural tube explants after 48 hrs of culture. Dissociated cells were initially passaged for three times and FACS sorted. Thus, total duration from harvest through FACS sorting was 12 days. In addition to the Wnt1-Cre;R26R-EGFP reporter previously employed previously, a PE (Phycoerythrin)-conjugated anti-CD44 antibody and APC (Allophycocyanin)-conjugated anti-Sca-1 antibody to isolate the stem cell population. 276 of EGFP/CD44/Sca-1 triple positive cells were clonally seeded on three 96 wells plates (a single cell per well) by means of an automated cell-seeding device. Initially, single cells were co-cultured with growth-inhibited STO feeder cells (approximately 3×10³ cells/cm²) in basal medium as described above. After four weeks, wells seeded with control feeder cells had no obvious cellular growth, while ten cranial neural crest-seeded wells had colonies of cells. These cells were trypsinized and passaged on fibronectin coated plates (without a feeder layer). Among them, 1 line was passageable clones (N16-1). Thus, the plating efficiency of clonal culture was 0.36%. A trend of marker expression was examined for 37 genes by RT-PCR. Obtained results matched to that of D7-1 with one exception. Higher expression of Snaill, a marker of neural crest, in N16-1 than D7-1 was observed. Subcloning of N16-1 was conducted following a protocol described above without CD44/Sca-1 selection. Out of 276 seeded N16-1 cells, 48 cells formed primary colonies. Eight passageable clones (N16-13, 14, 15, 16, 114, 117, 119, and 120) were obtained. Multipotency can be established, e.g., the ability to develop into osteoblasts, chondrocytes, smooth muscle cells, adipocytes, neurons and glial cells using methods described herein and known in the art.

### RT-PCR

QIAGEN RNeasy Kit was used to purify RNA samples. DNase treatment was performed by following to manufactures protocol. 2 μg RNA template was used for 40 μl scale of RT-reaction by Super script III (Invitrogen). cDNA was amplified by PCR program with 30 cycle reactions. β-actin was used as internal control. PCR products were run on 2% agarose gel and intensity of PCR product was quantified by NIH imageJ.

### Jak Inhibitor Treatment

AG490 (Sigma Chemical Co., St Louis, Mo., USA) was reconstituted in dimethyl sulfoxide (DMSO) and stored at −20° C. A stock concentration was 10 mM. Cranial tissues of Wnt1-Cre;R26R;EGFP mouse at E8.5 were dissociated by 0.025% trypsin and 1 mg/ml collagenase in DPBS. Then, neural crest cells labeled with Wnt1-Cre;R26R;EGFP were sorted by means of FACS and used for Jak inhibitor treatment. D7-1 or primary cultured cranial neural crest cells were seeded to fibronectin coated plate at a density of 20000 or 12700 cell/cm² respectively, and incubated for three days in basal medium as described above, added with either control DMSO, 10 μM AG490, or 20 μM AG490. Culture medium was changed daily. Alive cell number was counted on each day for D7-1 and third day for primary cultured cells.

### Example 8

### Characterization of Clone N16-1

Clone N16-1 was further characterized. As compared to clone D7-1, when the clone was cultured under conditions conductive to osteogenic differentiation, (short term culture, see above), the cells did not differentiate into osteoblasts in 24 hours unlike clone D7-1. The clone may differentiate under longer culture conditions.

Clone N16-1 cells also does not differentiate into smooth muscle, unlike D7-1. Instead, the clone shows remarkably reduced viability. Applicants also note that the response of mass cultured cell lines (09-1 and i10-1) is very similar to that of D7-1.

### Results and Conclusions

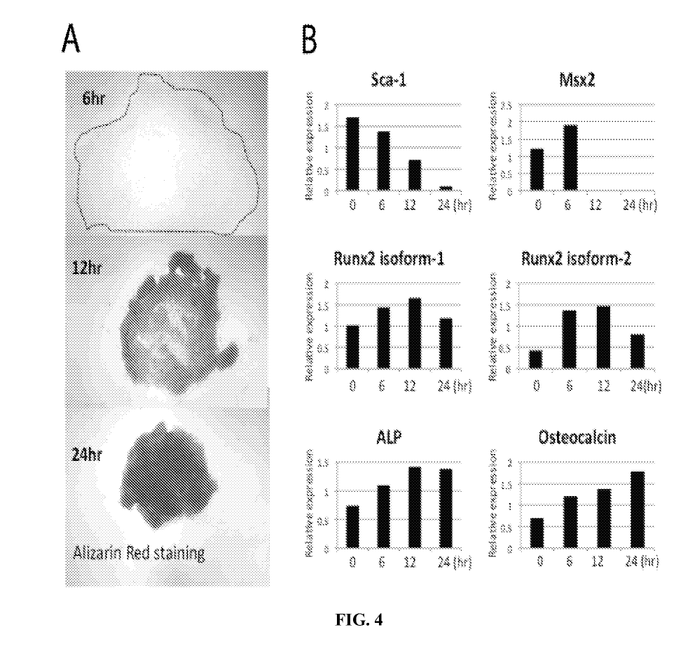

Applicants provide herein a cranial neural crest stem cell (craNCSC) and culture condition that allow the cells to grow as a homogeneous clonal line. The stem cell marker CD44 and Sca-1 are highly expressed in undifferentiated craNCSC clone D7-1. These markers are also expressed in mass cultured cranial neural crest (Chung et al. (2009)). Whether or not if they uniquely represent undifferentiated status of craNCSC was analyzed by performing 6 hrs, 12 hrs, and 24 hrs hanging-drop culture. Osteogenic differentiation of D7-1 (FIG. 4A) was induced. RNA from the cells at each time points and measured level of CD44 and Sca-1 as well as osteogenic marker expression by RT-PCR analysis. Importantly, a sharply declined Sca-1 mRNA expression during the course of experiments (FIG. 4B) was found. On the other hand, CD44 show a transient up-regulation at 12 hrs (not shown), and slight reduction at 24 hrs. Thus, CD44 is likely expressed in osteoprogenitors in addition to the undifferentiated craNCSC and it will not serve as ideal craNCSC marker. Therefore, Sca-1 is a potential cell surface antigen which defines undifferentiated status of craNCSC.

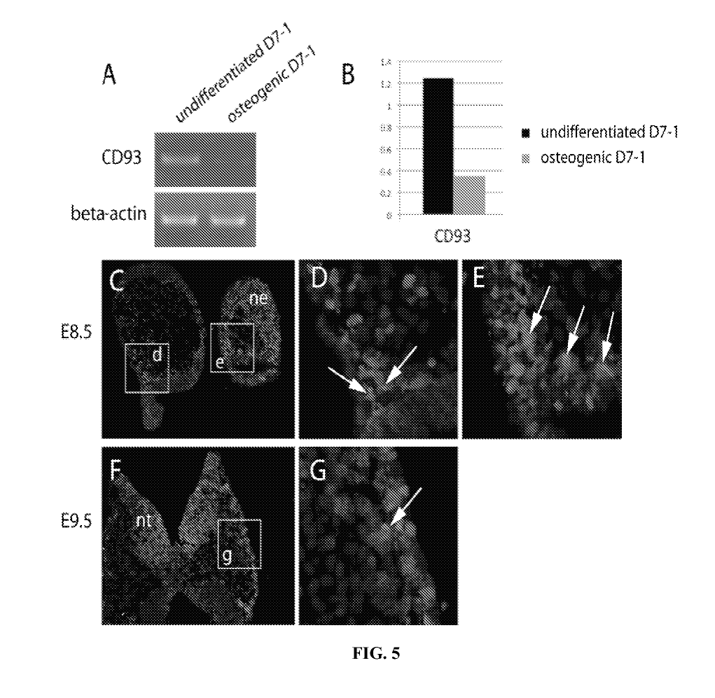

Next, with an aim of searching novel cell surface antigen(s) that define craNCSC, whole genome transcriptome analysis was preformed. Candidate genes that define craNCSC as described in below, were identified. These include, but are not limited to, CD81, CD9, CD34, CD47, CD38, CD200r, CD276, CD14, CD93 (AA4.1), CD274, and CD205. Next, Applicants identified antigen(s) that are uniquely expressed in undifferentiated craNCSC. By conducting RT-PCR, transcripts levels of these genes in undifferentiated D7-1 versus differentiated D7-1 cultured in osteogenic medium for one month were obtained. β-actin was used to normalize amount of input RNA. As results, expression of CD93 was significantly reduced in differentiated D7-1 by 3.6 fold (FIG. 5A and 5B). Thus, CD93 is an additional candidate to define undifferentiated craNCSC.

CD93 is known to be expressed in hematopoietic stem cell as well as in endothelial cells in developing embryo (Petrenko et al.(1999)). However, to the best of Applicant's knowledge, its expression in any neural crest population has not been described before. Thus, Applicants determined whether developing cranial neural crest express this cell surface antigen. E8.5 and E9.5 mouse neural crest cell were labeled with EGFP by means of Wnt1-Cre;R26R reporter system (Chai, et al. (2000), Jiang et al. (2000), Belteki et al. (2005)). Cryosections of these embryos were subjected to immunofluorescence with a PE (Phycoerythrin)-conjugated anti-CD93 antibody. Consistent with previous reports, Applicants observed CD93 expression in the endothelial cells. In addition, Applicants also found an expression of CD93 in small subset of both premigratory and migratory cranial neural crest cells at E8.5 (FIG. 5C, D, and E) . By E9.5, expression of CD93 in neural crest has become more restricted. Very few migratory neural crest cells express CD93 (FIG. 5F and G). These observations agree with our RT-PCR results suggesting only undifferentiated craNCSC maintain an expression of CD93. Therefore, without being bound by theory, CD93 positive cranial neural crest cell is believed to represent a stem cell fraction. Thus, Sca-1 and CD93 can be used as tools to isolate pure stem cell population from mass cranial neural crest in developing or adult craniofacial apparatus as well as differentiated derivatives of pluripotent stem cells.

Thus, these finding enable the use of APC conjugated anti-Sca-1 and PE conjugated anti-CD93 antibodies with an aim to isolate and enrich a stem cell fraction from developing mouse cranial neural crest. To perform the method and isolate the cells, cells from Wnt1-Cre;R26R;EGFP labeled E8.5 mouse heads are subjected to FACS sorting with above mentioned antibodies. Cells are obtained by either directory trypsinizing embryonic tissues or harvesting migratory cells from 48 hrs cultured neural tube explants. EGFP positive neural crest cell can be gated to (1) CD93-PE positive, (2) Sca-1-APC positive, (3) CD93-PE and Sca-1-APC double positive, or (4) double negative fraction. Then, the cells are cultured as both mass and clonal culture format. Marker gene expression and differentiation capability of these cells can be evaluated. CD93-PE and Sca-1-APC double positive subgroup can be cultured and grown in Applicants' basic culture condition (as described herein), and these cells show stem cell phenotype.

Parallel to above mentioned experiments, unexamined CD markers and cell surface antigens are also investigated to determine if they can also serve as undifferentiated craNCSC markers.

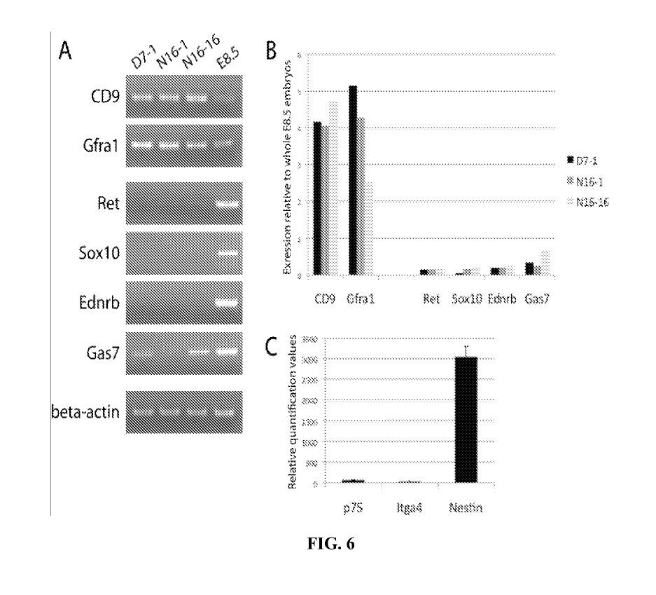

p75 (Ngfr) and Itga4 (Alpha 4 integrin) are cell surface antigens used to isolate trunk neural crest stem cells (trunkNCSC) and its relative, gut neural crest stem cells (gutNCSC) (Stemple and Anderson (1992); Bixby et al. (2002)). The level of transcript of these genes in D7-1 were investigated. Surprisingly, however insignificant levels of expression of them in craNCSC were found (FIG. 6C). Thus, it is speculated that conventional marker for trunkNCSC and gutNCSC may not be suitable for defining craNCSC.

### Novelty of Cranial Neural Crest Stem Cells

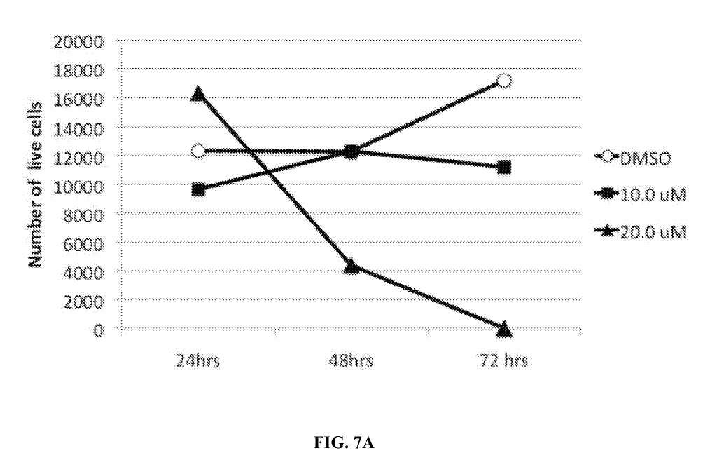

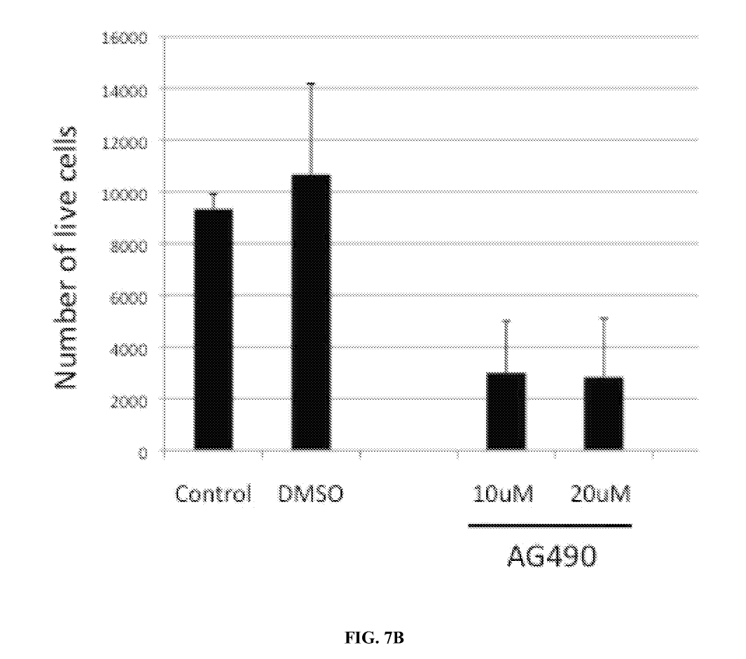

Low level expression of p75 and Itga4 in D7-1 prompted the question whether cranial neural crest stem cell is just a simple counter part of trunkNCSC in craniofacial tissues. Previously, another group searched for a characteristic markers highly expressed in gutNCSC (Iwashita et al. (2003)). By comparing whole genome transcriptome of E14.5 rat embryos and freshly isolated gutNCSC from embryos at same stage, they found specific markers prominently expressed in gutNCSC. These gutNCSC markers include Ret, Cd9, Sox10, Gfra1, Gas7, and Ednrb. PCR primers were designed to see if they are also abundant in craNCSC. RT-PCR analysis was performed for RNA samples extracted from whole E8.5 mouse embryo and 2 independent craNCSC clones as well as 1 subclone derived from one of these clones (clone D7-1, N16-1 and N16-16). As results, mRNA of Ret, Sox10, Gas7, and Ednrb, were either significantly reduced or unable to detect in craNCSC (FIG. 7A and 7B), see Table 3. Thus, clear dissimilarities between craNCSC and gutNCSC are evident. On the other hand, a similarity was found among them. As it has seen in gutNCSC, expression level of CD9 and Gfral is higher in craNCSC compare to their origin of whole embryonic tissues (FIGS. 7A and 7B). TrunkNCSC marker nestin is expressed in craNCSC at significant level was also found (FIG. 7C). Therefore, some of the characteristics of cranial and trunk neural crest stem cells are in common.

In summary, remarkably distinctive marker gene expression profile with a partial overlapping in craNCSC and gutNCSC suggests that they are related cell-type, but they are not simple counter part in different tissues, rather, they are discrete subpopulation of developing neural crest.

### The Jak-STAT Pathway

Signal transducers and activators of transcription (STATs) mediate a wide variety of cellular responses to cytokines and growth factors. Janus kinases (JAKs) dependent phosphorylation activates STATs then they dimerize and translocate to the nucleus where they initiate transcription of target genes. This pathway is known to control cell survival, proliferation, differentiation and migration (Levy and Darnell (2002); Androutsellis-Theotokis et al. (2006)). Leukemia inhibitory factor WO belongs to the IL-6 cytokine family that act on the LIFR/gp130 receptor complex to signal via the JAK-STAT pathway.

Applicants hypothesized that LIF stimulated the JAK-STAT pathway promotes sustainable growth of cranial neural crest stem cell (craNCSC). Based on this hypothesis, LIF supplemented medium was tested to achieve long-term growth of craNCSC. This medium was also conditioned by gamma-irradiated STO feeder cells which is known to secrete LIF (Williams et al. (1988)). Supporting this hypothesis, Applicants have established long-term culture of craNCSC with this medium. In addition, by performing whole genome expression analysis, a significant transcriptional level of the major components of the JAK-STAT pathway in craNCSC clone D7-1 were found. These include LIFR, gp130, JAK1, JAK2, STAT1, STAT3, and STAT5. This suggests the JAK-STAT pathway is active in craNCSC. However, a direct evidence for the requirement of the JAK-STAT function in these cells has not been provided yet.

The hypothesis that phosphorylation of STAT3 by JAK kinase is essential for the maintenance of craNCSC was also investigated. JAK inhibitor AG490 was used for this study. AG490 is synthetic PTK inhibitor with anti-JAK2 activity. When cells are treated with this chemical, STAT3 will remains inactive due to inhibition of JAK2 kinase. AG490 prevents the growth of a human B-precursor leukemic cell line by inducing apoptosis (Meydan et al. (1996)), while it causes no obvious effect for the cells which are independent from the JAK-STAT signal (Ding et al. (2008)). If craNCSC require the JAK-STAT signal for its maintenance, treatment of AG490 will cause overt effects. 10 μM and 20 μM AG490 treatment against D7-1 was performed for three days and alive cell number was counted on each day. Importantly, 20 μM AG490 treatment was found to result in significant reduction of the viable cells over the time course, while 10 μM AG490 treatment had a smaller effect (FIG. 7A). It is known that 80 μM AG490 treatment does not cause general toxicity to the STAT3 negative cells (Ding et al. (2008)). Therefore, Applicants concluded an observed high mortality of D7-1 was due to inhibition of the JAK-STAT signal by AG490. Dose dependent susceptibility of D7-1 against AG490 was also evident.

These experiments were done with a long-term cultured craNCSC clone, D7-1, which has been treated with LIF during entire culture process. Therefore, it is possible that D7-1 cells has been poised to LIF and adopted to the JAK-STAT dependent culture condition. To evaluate this, Applicants freshly isolated Wnt1-Cre;R26R;EGFP positive cranial neural crest cells from E8.5 mouse embryos by FACS sorting and treated them with AG490 for three days. If JAK2 activity is dispensable to these primary neural crest, chemical treatment would not cause any apparent effects. Intriguingly, however, Applicants observed high mortality of JAK2 inhibitor treated cells at dose of either 10 μM and 20 μM (FIG. 8B). Thus, JAK2 activity is required for the cell survival in both long-term or primary cultured cranial neural crest.

These data strongly suggest that the JAK-STAT pathway has an essential role to maintain the stemness of craNCSC. Currently, Applicants are aiming to further address which cellular properties and molecular mechanisms are controlled by the JAK-STAT. Those knowledge will enable us to control craNCSC growth and differentiation in more effective ways.

Applicants will look to additional aspects of JAK-STAT dependent cellular behaviors of craNCSC. Applicants hypothesize that the JAK-STAT is not only required for craNCSC survival, but also for its self-renewal, proliferation, migration, and lineage-determination. In order to prove this model, the effect of JAK and STAT inhibitor at low-dose which will not cause high cells mortality will be investigated.

Western blotting and immunofluorescence will also confirm that high cell mortality caused by AG490 is due to reduced or lack of activation of STAT3 and/or other STAT family. Applicants are assuming that a reduced level of STAT3 phosphorylation at Tyr705 in AG490 treated cells triggers the programmed cell death. However, it is also possible that additional STAT families are required for maintenance of craNCSC. Applicants will further examine this possibility by treating the cells with chemical inhibitors of different STATs (see below). Applicants hypothesize that some of them will cause an equivalent effects of AG490. siRNA gene targeting of different STATs will confirm these results.

STAT1 inhibitors;

| | |
|---|---|
| fludarabine | (Frank et al. (1999) Nat. Med. |
| | 5(4): 444-7) |
| 5′-deoxy-5′-(methylthio)- | (Shen and Lentsch, 2004) |
| adenosine (MTA) | |

STAT3 inhibitors;

| | |
|---|---|
| Stattic | (Schust et al. (2006) Chem. Biol. |
| | 13(11): 1235-42) |
| NSC 74859 | (Siddiquee et al. (2007) Proc. Natl. |
| | Acad. Sci. USA 104(18): 7391-6) |

STAT5 inhibitor;

| | |
|---|---|
| Lestaurtinib (CEP701) | (Hexner et al. (2008) Blood |
| | 111(12): 5663-71) |

The functions of STAT3 in developing mouse neural crest will also be investigated. First, Applicants will perform in situ hybridization to determine the mRNA expression pattern of JAK-STAT components in E8.5 through E10.5 mouse embryos. Applicants will also study phosphorylation status of STAT3 in embryonic neural crest cells by immunofluorescence. Then, Applicants will analyze loss of function phenotype of STAT3 in mouse neural crest. Wnt1-Cre transgenic animals will be crossed into STAT3 flox/flox conditional mutants. Conventional STAT3 homozygote mutant mice exhibit embryonic lethality before E7.5 (Takeda et al. (1997)). Thus, only conditional mutant allele will enable Applicants to study the role of STAT3 in mouse at E8.5 and later stages.

Applicants hypothesize that dramatic phenotype in the mutant mice due to ranges of neural crest anomalies in cell production, proliferation, survival, patterning, and differentiation will be observed.

Applicants will also identify downstream target(s) of JAK-STAT in craNCSC. Reported potential targets positively controlled by STAT3 include Ccnd1, Hsp90, Cox2, Vim, Hif1α, Myc, Mcl1, Birc5, Vegf, Twist1, Cxcl12, Il-11, Icam1, and Fgf2 (Yu et al. (2009)). Importantly, these genes have a significant level of transcripts in craNCSC clone D7-1. Thus, in theory, STAT3 can function and activate transcription of those downstream target genes in D7-1. Among the candidates, Fgf2, CyclinD1, Myc, and Twist1are genes of primary interest since they are involved in neural crest development which are believed to be key effecters of JAK2-STAT3 signal and activation of these genes is essential to craNCSC development.

Applicants have described about a minimal set of neural crest marker expression found in D7-1, a clonal line of cranial neural crest stem cell (craNCSC). Those genes are AP-2α, Twist1), Snail2, Msx2, Dlx1, Dlx2, Pax3, Ets1, Foxc1, Crabp1, and Cadherin6. Up to now, Applicants identified additional neural crest markers expressed in D7-1; Cnbp, Eif4a2, Ets2, Gli3, Myc, Sox4, Sox9, Tcof1, Cdh11, Cdc4, Fbxw7, Fmr1, Fn1, Fxr1, Fzd3, Fzd6, Fzd7, Gdnf, Id2, Meis1, Myo10, Notch1, Nrp1, Nrp2, Rhob, Robo1, Sulf2, and Zic2.

Recently, Bronner-Fraser's group has reported a group of genes that could serve as novel neural crest markers (Adams et al. (2008)). Among them, 76 genes have obvious homologue in mouse, and we found 61 genes (80.3% of total) are expressed in D7-1 at significant level. Those are Adh5, Akap1, Aldh9a1, Ankrd17, Atpla1, Basp1, Bid, Cachd1, Ccar1, Ccnb2, Ciapin1, Col4a5, Ctcf, Ctnna1, Ctsb, Ddx23, Elk3, Ewsr1, G3bp1, Gart, Glg1, Gnl2, Gtf2e1, Gstcd, H3f3b, Heph, Hk2, Hnrnpa2b1, Hnrnpm, Hplbp3, Ilf2, Ilf3, Ipo9, Ktn1, Lmnb2, Macf1, Mcm2, Mcm5, Mkrn1, Msh6, Nes, Nf2, Nsun5, Psmd3, Ptprf, Pxn, Rbm4, Rcc2, Rnh1, Sec14l1, Srebf2, Srf, Taldo1, Tcf20, Thoc5, Tnrc18, Tpd5212, Tpm3, Trio, Vav2, and Whsc1l1.

On the other hand, significantly low level expression of non-neural crest markers was found. For instance, all family members of Hox gene, endoderm marker (Sox17, Afp, and Pdx1), and mesoderm marker (Mesp1, Mesp2, T, Gata4, Gsc, and Noda1) appeared to have greatly lower level transcriptional activity than above mentioned neural crest markers. In addition, expression of terminally differentiated cell marker was found to be also insignificant. Those include markers for osteogenic (osterix, ALP, osteocalcin, and bone sialoprotein), chondrogenic (Comp), smooth muscle (αSMA and calponin), myogenic (MyoD, Myogenin, Myf5, and MRF4), neuronal (neuron-specific class III β-tubulin and Peripherin) and Schwann cell (S100b, MBP and GFAP). These results illuminate a specificity of transcriptional activity of neural crest marker in D7-1.

### Example 8

### Endothelin Pathway

Endothelin1 (Edn1) signaling and MEF2C act as upstream regulator of Dlx5, Dlx6 and Hand2 in the branchial arches. This pathway is required for proper craniofacial development of vertebrate species (Verzi et al. (2007); Miller et al. (2007)). Intriguingly, a set of genes involved in this pathway including Ednl, endothelin receptor type A and B (Ednra and Ednrb), MEF2C, Dlx5, Dlx6 and Hand2 is poorly expressed in cranial neural crest stem cell D7-1. This suggests that Ednl pathway is actively repressed in craNCSC and down-regulation of Edn signal is required for the maintenance of stemness. Applicants believe that Endothelin (Edn) signaling has an essential role in promoting differentiation of stem cell population in cranial neural crest. By manipulating Edn signals, one can govern a balance between undifferentiated and differentiated status of cranial neural crest stem cells. craNCSC will remain as undifferentiated stem cells and exert enhanced growth if treated with Edn pathway inhibitor such as Ednra antagonist BQ-123, ABT-627, and Ednrb antagonist A-192621 as well as Ednra and Ednrb antagonist A-182086 (Yin et al. (2003)). As opposing effects, exogenous Edn1 treatment will trigger cell differentiation of craNCSC. This cytokine can be used to achieve an efficient production of cranial neural crest derivatives including osteoblast, chondrocyte, smooth muscle, adipocyte, neuronal cell, glial cell, or other cell-type.

### Example 9

### HIF1α Related (Hypoxia Culture may Enhance craNCSC)

It has been shown that trunk NCSC plating efficiency can be improved by hypoxia culture condition. It is possible that craNCSC also respond similarly under this culture condition. Supporting this notion, Applicants have found significant level of HIF1α expression. HIF1α is known to be active in cells that favor hypoxia condition.

### Example 10

### Regulators of Chromatin Modifications

The chromatin modifiers may play important role for maintaining a particular chromatin structure of craNCSC. Very recently, Wysocka's group has found that Chd7, one of the member of Trithorax group protein, interacts with chromatin remodelling complexes of the SWI/SNF family to activate neural crest specific genes, Twist1 and Sox9, in neural crest-like cells induced from human ES cell (Bajpai et al. (2010)). This result also is consistent with a loss of function phenotype of Chd7 in xenopus embryo (Bajpai et al. (2010)). A unique combination of modified chromatin signify an essential characteristics of particular stem cell line as it maintains specific gene expression profile (Bernstein et al. (2006)).

By analyzing microarray data, Applicants found that craNCSC clone D7-1 expresses wide varieties of genes that encode chromatin-modifiers (Schuettengruber et al. (2007); Simon and Kingston (2009)). First, the Polycomb group (PcG) expression was found. A high level of expression of core components of Polycomb repressive complex 1 and 2 (PRC1 and PRC2) as well as their interacting proteins was also observed. PRC2 methylates histone H3 on Lys27 (H3K27) and PRC1 exert chromatin silencing. Second, expression of members of the Trithorax group (TrxG) which has opposing role of PRC1 and PRC2. TrxG induces histone H3 Lys4 (H3K4) methylation required for a transcriptional activation of genes repressed by silent chromatin also was found. Finally, expression of genes coding chromatin remodeling proteins was further found. Following list includes but not limited chromatin-modifiers found in craNCSC.

TABLE 4
PRC1; Ring1B, Cbx2, Cbx4, Cbx6, Cbx7, Cbx8, Phc1, Phc2, Phc3,
Bmi1, Mel18, Nspc1
PRC2; Ezh1, Ezh2, Eed, Suz12, RBAP46
PRC1 and 2 related proteins; Asxl2, Epc1, MBLR, YY1, Jarid2
TrxG; Setd1a, Wdr5, Ash2l, Rbbp5, BRM, BRG1, SNF2L, BAF250
Chromatin remodeling proteins; Chd1, Chd3, Chd4, Chd8

Thus, this unique combination of chromatin-modifier complexes is required for a distinguished pattern of chromatin modification that defines transcriptome and stemness of craNCSC. Whole genome chromatin immunoprecipitation (ChIP) analysis with antibodies against H3K4 and H3K27 will reveal specific chromatin signature in craNCSC. Applicants hypothesize that this pattern will be dramatically altered when cells are exposed to the condition that triggers their differentiation. Furthermore, ChIP assay against core components of chromatin-modifier complexes, such as Ezh2, Suz12, and BRG1, combined with microarray assay (ChIP on chip) will lead to understanding of the molecular mechanisms that control the balance between a repressed or an active state chromatin in craNCSC.

Unless otherwise defined, all technical and scientific terms used herein have the same meaning as commonly understood by one of ordinary skill in the art to which this invention belongs. All nucleotide sequences provided herein are presented in the 5′ to 3′ direction.

Thus, it should be understood that although the present invention has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

It is to be understood that while the invention has been described in conjunction with the above embodiments, that the foregoing description and examples are intended to illustrate and not limit the scope of the invention. Other aspects, advantages and modifications within the scope of the invention will be apparent to those skilled in the art to which the invention pertains.

### REFERENCES

Adams M. S., Gammill L. S., Bronner-Fraser M. (2008) Discovery of transcription factors and other candidate regulators of neural crest development. Dev. Dyn. 237(4), 1021-33.
Androutsellis-Theotokis A., Leker R. R., Soldner F., Hoeppner D. J., Ravin R., Poser S. W., Rueger M. A., Bae S. K., Kittappa R., McKay R .D. (2006) Notch signalling regulates stem cell numbers in vitro and in vivo. Nature 442(7104), 823-6.
Bajpai R., Chen D. A., Rada-Iglesias A., Zhang J., Xiong Y., Helms J., Chang C. P., Zhao Y., Swigut T., Wysocka J. (2010) CHD7 cooperates with PBAF to control multipotent neural crest formation. Nature 463(7283), 958-62.
Baroffio, A., Dupin, E. and Le Douarin, N. M. (1988). Clone-forming ability and differentiation potential of migratory neural crest cells. Proc Natl Acad Sci USA 85, 5325-9.
Bernstein B. E., Mikkelsen T. S., Xie X., Kamal M., Huebert D. J., Cuff J., Fry B., Meissner A., Wernig M., Plath K., Jaenisch R., Wagschal A., Feil R., Schreiber S. L., Lander E. S. (2006) A bivalent chromatin structure marks key developmental genes in embryonic stem cells. Cell 125(2):315-26.
Bixby S., Kruger G. M., Mosher J. T., Joseph N. M., Morrison S. J. (2002). Cell-intrinsic differences between stem cells from different regions of the peripheral nervous system regulate the generation of neural diversity. Neuron 35(4), 643-56.
Calloni, G. W., Glavieux-Pardanaud, C., Le Douarin, N. M. and Dupin, E. (2007). Sonic Hedgehog promotes the development of multipotent neural crest progenitors endowed with both mesenchymal and neural potentials. Proc Natl Acad Sci USA 104, 19879-84.
Calloni, G. W., Le Douarin, N. M. and Dupin, E. (2009). High frequency of cephalic neural crest cells shows coexistence of neurogenic, melanogenic, and osteogenic differentiation capacities. Proc Natl Acad Sci USA 106, 8947-52.
Chai, Y., Jiang, X., Ito, Y., Bringas, P., Jr, Han, J., Rowitch, D. H., Soriano, P., McMahon, A. P. and Sucov, H. M. (2000). Fate of the mammalian cranial neural crest during tooth and mandibular morphogenesis. Development 127,1671 -1679.
Chai, Y. and Maxson, R. E., Jr. (2006). Recent advances in craniofacial morphogenesis. Dev Dyn 235, 2353-75.
Chung I H, Yamaza T, Zhao H, Choung P H, Shi S, Chai Y. (2009) Stem cell property of postmigratory cranial neural crest cells and their utility in alveolar bone regeneration and tooth development. Stem Cells 27, 866-77.
Cowan, C. M., Shi, Y. Y., Aalami, O. O., Chou, Y. F., Mari, C., Thomas, R., Quarto, N., Contag, C. H., Wu, B. and Longaker, M. T. (2004). Adipose-derived adult stromal cells heal critical-size mouse calvarial defects. Nat Biotechnol 22, 560-7.
D'Addona, A. and Nowzari, H. (2001). Intramembranous autogenous osseous transplants in aesthetic treatment of alveolar atrophy. Periodontol 2000 27, 148-61.
da Silva Meirelles, L., Chagastelles, P. C. and Nardi, N. B. (2006). Mesenchymal stem cells reside in virtually all post-natal organs and tissues. J Cell Sci 119, 2204-13.

Delfino-Machin, M., Chipperfield, T. R., Rodrigues, F. S. and Kelsh, R. N. (2007). The proliferating field of neural crest stem cells. Dev Dyn 236, 3242-54.
Ding B. B., Yu J. J., Yu R. Y., Mendez L. M., Shaknovich R., Zhang Y., Cattoretti G., Ye B. H. (2008) Constitutively activated STAT3 promotes cell proliferation and survival in the activated B-cell subtype of diffuse large B-cell lymphomas. Blood 111(3), 1515-23.
Fernandes, K. J., McKenzie, I. A., Mill, P., Smith, K. M., Akhavan, M., Barnabe-Heider, F., Biernaskie, J., Junek, A., Kobayashi, N. R., Toma, J. G. et al. (2004). A dermal niche for multipotent adult skin-derived precursor cells. Nat Cell Biol 6, 1082-93.
Frank D. A., Mahajan S., Ritz J. (1999) Fludarabine-induced immunosuppression is associated with inhibition of STAT1 signaling. Nat. Med. 5(4), 444-7.
Hexner E. O., Serdikoff C., Jan M., Swider C. R., Robinson C., Yang S., Angeles T., Emerson S. G., Carroll M., Ruggeri B., Dobrzanski P. (2008) Lestaurtinib (CEP701) is a JAK2 inhibitor that suppresses JAK2/STATS signaling and the proliferation of primary erythroid cells from patients with myeloproliferative disorders.Blood 111(12), 5663-71
Iwashita, T., Kruger, G. M., Pardal, R., Kiel, M. J. and Morrison, S. J. (2003). Hirschsprung disease is linked to defects in neural crest stem cell function. Science 301, 972-6.
Jiang, X., Rowitch, D. H., Soriano, P., McMahon, A. P. and Sucov, H. M. (2000). Fate of the mammalian cardiac neural crest. Development 127,1607 -1616.
Kubota, H., Avarbock, M. R. and Brinster, R. L. (2004b). Growth factors essential for self-renewal and expansion of mouse spermatogonial stem cells. Proc. Natl. Acad. Sci. USA 101,16489 -16494.
Leucht, P., Kim, J. B., Amasha, R., James, A. W., Girod, S. and Helms, J. A. (2008). Embryonic origin and Hox status determine progenitor cell fate during adult bone regeneration. Development 135, 2845-54.
Levy D. E., Darnell J. E. Jr. (2002) Stats: transcriptional control and biological impact. Nat. Rev. Mol. Cell Biol. 3(9), 651-62.
Meydan N., Grunberger T., Dadi H., Shahar M., Arpaia E., Lapidot Z., Leeder J. S., Freedman M., Cohen A., Gazit A., Levitzki A., Roifman C. M. (1996) Inhibition of acute lymphoblastic leukaemia by a Jak-2 inhibitor. Nature 379(6566), 645-8.
Morrison, S. J., White, P. M., Zock, C. and Anderson, D. J. (1999). Prospective identification, isolation by flow cytometry, and in vivo self-renewal of multipotent mammalian neural crest stem cells. Cell 96, 737-49.
Pardal, R., Ortega-Saenz, P., Duran, R. and Lopez-Barneo, J. (2007). Glia-like stem cells sustain physiologic neurogenesis in the adult mammalian carotid body. Cell 131, 364-77.
Rovasio, R. A., Delouvee, A., Yamada, K. M., Timpl, R. and Thiery, J. P. (1983). Neural crest cell migration: Requirements for exogenous fibronectin and high cell density. J. Cell Biol 96, 462-473.
Santagati, F. and Rip, F. M. (2003). Cranial neural crest and the building of the vertebrate head. Nature reviews. Neuroscience 4, 806 -818.
Schuettengruber B., Chourrout D., Vervoort M., Leblanc B., Cavalli G. (2007) Genome regulation by polycomb and trithorax proteins. Cell 128(4), 735-45.
Schust J., Sperl B., Hollis A., Mayer T. U., Berg T. (2006) Stattic: a small-molecule inhibitor of STAT3 activation and dimerization. Chem. Biol. 13(11), 1235-42.
Siddiquee K., Zhang S., Guida W. C., Blaskovich M. A., Greedy B., Lawrence H. R., Yip M. L., Jove R., McLaughlin M. M., Lawrence N. J., Sebti S. M., Turkson J. (2007) Selective chemical probe inhibitor of Stat3, identified through structure-based virtual screening, induces antitumor activity. Proc. Natl. Acad. Sci. USA 104(18), 7391-6.
Simon J. A., Kingston R. E. (2009) Mechanisms of polycomb gene silencing: knowns and unknowns.Nat. Rev. Mol. Cell Biol. 10(10):697-708.
Stemple, D. L. and Anderson, D. J. (1992). Isolation of a stem cell for neurons and glia from the mammalian neural crest. Cell 71, 973-85.
Takeda K., Noguchi K., Shi W., Tanaka T., Matsumoto M., Yoshida N., Kishimoto T., Akira S. (1997) Targeted disruption of the mouse Stat3 gene leads to early embryonic lethality. Proc. Natl. Acad. Sci USA 94(8), 3801-4.
Thomas, S., Thomas, M., Wincker, P., Babarit, C., Xu, P., Speer, M. C., Munnich, A., Lyonnet, S., Vekemans, M. and Etchevers, H. C. (2008). Human neural crest cells display molecular and phenotypic hallmarks of stem cells. Hum Mol Genet 17, 3411-25.
Ting M C, Wu N L, Roybal P G, Sun J, Liu L, Yen Y, Maxson R E Jr. (2009) EphA4 as an effector of Twist1in the guidance of osteogenic precursor cells during calvarial bone growth and in craniosynostosis. Development 136, 855-864.
Trentin, A., Glavieux-Pardanaud, C., Le Douarin, N. M. and Dupin, E. (2004). Self-renewal capacity is a widespread property of various types of neural crest precursor cells. Proc Natl Acad Sci USA 101, 4495-500.
Williams R. L., Hilton D. J., Pease S., Willson T. A., Stewart C. L., Gearing D. P., Wagner E. F., Metcalf D., Nicola N. A., Gough N. M. (1988) Myeloid leukaemia inhibitory factor maintains the developmental potential of embryonic stem cells. Nature 336(6200), 684-7.
Wobus, A. M., Guan, K., Yang, H. T. and Boheler, K. R. (2002). Embryonic stem cells as a model to study cardiac, skeletal muscle, and vascular smooth muscle cell differentiation. Methods Mol. Biol. 185,127 -156.
Xu, C., Inokuma, M. S., Denham, J., Golds, K., Kundu, P., Gold, J. D. and Carpenter, M. K. (2001). Feeder-free growth of undifferentiated human embryonic stem cells. Nat. Biotechnol. 19, 971-974.
Yin J. J., Mohammad K. S., Käkönen S. M., Harris S., Wu-Wong J. R., Wessale J. L., Padley R. J., Garrett I. R., Chirgwin J. M., Guise T. A. (2003) A causal role for endothelin-1 in the pathogenesis of osteoblastic bone metastases. Proc. Natl. Acad. Sci. USA 100(19):10954-9.
Yoshida, S., Shimmura, S., Nagoshi, N., Fukuda, K., Matsuzaki, Y., Okano, H. and Tsubota, K. (2006). Isolation of multipotent neural crest-derived stem cells from the adult mouse cornea. Stem Cells 24, 2714-22.
Yu H., Pardoll D., Jove R. (2009) STATs in cancer inflammation and immunity: a leading role for STAT3. Nat. Rev. Cancer 9(11), 798-809.

1. An isolated self-renewable cranial neural crest stem cell.

2. The isolated cranial neural crest stem cell of claim 1, wherein the isolated cranial neural crest stem cell is one or more of: multipotent; capable of differentiation into at least one or two cell type(s) selected from the group of an osteoblast, a chondrocyte, a smooth muscle cell a glial cell, a neuronal cell or an adipocyte.

3. (canceled)

4. (canceled)

5. The isolated cranial neural crest stem cell of claim 2, wherein the isolated cranial neural crest stem cell is capable of differentiation into at least three of the cell types.

6.-8. (canceled)

9. The isolated cranial neural crest stem cell of claim 1, wherein the isolated cranial neural crest stem cell expresses one or more marker of the group CD44, Sca-1, nestin, AP-2α, Twist1, Snail1, Snail2, CD93 or EGFP.

10. The isolated cranial neural crest stem cell of claim 9, wherein the isolated cranial neural crest stem cell further expresses one or more marker of the group AP-2α, Twist1, Snail2, Msx2, Dlx1, Dlx2, Pax3, Ets1, Foxc1, Crabp1, and Cadherin6.

11. The isolated cranial neural crest stem cell of claim 9 or 10, wherein the isolated cranial neural crest stem cell further expresses one or more marker of the group D7-1; Cnbp, Eif4a2, Ets2, Gli3, Myc, Sox4, Sox9, Tcof1, Cdh11, Cdc4, Fbxw7, Fmr1, Fn1, Fxr1, Fzd3, Fzd6, Fzd7, Gdnf, Id2, Meis1, Myo10, Notch1, Nrp1, Nrp2, Rhob, Robo1, Sulf2, and Zic2.

12. The isolated cranial neural crest stem cell of claim 1, wherein the isolated cranial neural crest stem cell expresses Sca-1 and at least one or more marker of the group CD44, nestin, AP-2α, Twist1, Snail1, Snail2, CD93 or EGFP.

13. The isolated cranial neural crest stem cell of claim 1, wherein the isolated cranial neural crest stem cell expresses Sca-1 and CD93 at least one or more marker of the group CD44, nestin, AP-2α, Twist1, Snail1, Snail2, or EGFP.

14. The isolated cranial neural crest stem cell of claim 13, wherein the isolated cranial neural crest stem cell further expresses one or more marker of the group Gfra1, CD81, CD9, CD34, CD47, CD38, CD200r, CD276, CD14, CD93 (AA4.1), CD274 or CD205.

15. The isolated cranial neural crest stem cell of claim 13 or 14, wherein the isolated cranial neural crest stem cell further expresses one or more marker of the group LIFR, gp130, JAK1, JAK2, STAT1, STAT3, or STAT5.

16. The isolated cranial neural crest stem cell of claim 15, wherein the isolated cranial neural crest stem cell further expresses one or more marker of the group Ccnd1, Hsp90, Cox2, Vim, Hif1α, Myc, Mcl1, Birc5, Vegf, Twist1, Cxcl12, Il-11, Icam1, or Fgf2.

17. The isolated cranial neural crest stem cell of claim 1, wherein the isolated cranial neural crest stem cell does not expresses or only expresses at a low level one or more marker of the group Ret, Sox10, Gas7 or Ednrb.

18. The isolated cranial neural crest stem cell or claim 1, wherein the isolated cranial neural crest stem cell does not expresses or only expresses at a low level one or more marker of the group Sox17, Afp, and Pdx1, Mesp1, Mesp2, T, Gata4, Gsc, Nodal or a terminal differentiation marker for osteogenic, chondrogenic, smooth muscle, myogenic, neuronal, or Schwann cell.

19. The isolated cranial neural crest stem cell of claim 1, wherein the isolated cranial neural crest stem cell can be passaged for a time selected from the group of for at least about 10 times; for at least about 30 times; for at least about 100 times; for at least about 1 month; for at least about 3 months; or for at least about 6 months.

20.-24. (canceled)

25. The isolated cranial neural crest stem cell of claim 1, wherein the isolated cranial neural crest stem cell is a mammalian cell.

26. An isolated clonal population of the isolated cranial neural crest stem cell of claim 1.

27. An isolated population of self-renewable multipotent cranial neural crest stem cells.

28. The isolated population of claim 27, wherein the cranial neural crest stem cells are capable of differentiation into at least three cell types selected from the group of an osteoblast cell, a chondrocyte, a smooth muscle cell, a glial cell, a neuronal cell or an adipocyte.

29. The isolated cranial neural crest stem cell of claim 1, further comprising an exogeneous agent.

30. The isolated neural crest stem cell or population of claim 29, wherein the agent is one or more of a small molecule, detectable label, antibody or a non-naturally occurring nucleic acid.

31. An substantially homogeneous population of isolated neural crest stem cells or populations of claim 1 or 29.

32. A method for expanding an isolated neural crest stem cell of claim 1, comprising contacting the cell with an effective amount of stem cell growth medium supplemented with from about 10% to about 20% Fetal Bovine Serum (FBS), thereby expanding the stem cell or population.

33. The method of claim 32, further comprising contacting the cell with from about 15 ng/ml to about 35 ng/ml bFGF.

34. The method of claim 33, wherein the stem cell growth medium further comprises from about 700 U to about 1300 U of LIF.

35. A cranial neural crest stem cell growth medium comprising stem cell growth medium supplement with from about 10% to about 20% FBS and optionally from about 15 ng/ml to about 35 ng/ml of bFGF.

36. The cranial neural crest stem cell growth medium of claim 35, further comprising from about 700 U to about 1300 U of LIF.

37. The growth medium of claim 35, further comprising one or more of Dulbecco's modified Eagle's medium (DMEM), about 0.1 mM MEM nonessential amino acids, about 0.1 mM sodium pyruvate, about 55 μM β-mercaptoethanol, about 100 units/ml penicillin, about 100 units/ml streptomycin or about 2 mM L-glutamine.

38. The growth medium of claim 36 conditioned by STO feeder cells.

39. The growth medium of claim 38 conditioned by STO feeder cells for at least about 24 hours.

40. The growth medium of claim 36, wherein the FBS is presented at a concentration of about 12% to about 17% or about 15%.

41. (canceled)

42. The growth medium of claim 36, wherein the bFGF is presented at a concentration from about 20 ng/m to about 30 ng/ml or about 25 nq/ml.

43. (canceled)

44. The growth medium of claim 36, wherein the LIF is presented at a concentration from about 700 U to about 1300 U or about 1000 U.

45. (canceled)

46. A method of culturing a cranial neural stem cell comprising growing a cranial stem cell in a growth medium of claim 36.

47. A population of cranial neural stem cells obtained by the method of claim 36.

48. The population of method of claim 47, wherein the population comprises a plurality of clonal self-renewable multipotent cranial neural crest stem cells.

49. A kit for use in culturing a cranial neural stem cell comprising an effective amount of the growth medium of claim 36 and instructions for use of the growth medium.

50. A method for ameliorating the symptoms of a CraNCSC treatable disease, condition or disorder in a subject in need thereof, comprising administering to the subject an effective amount of the isolated cranial neural crest stem cell of claim 1, thereby ameliorating the symptoms in the subject.

51. A method for treating a subject in need thereof, comprising administering to the subject an effective amount of the isolated cranial neural stem cell of claim 1, thereby treating the subject.

52. The method of claim 50 or 51, wherein the disorder is one or more a critical size defect in cranial skeletal bone, skeletal tissue, joints or replacement or healing of bone or cartilage.

53. A method for identifying an agent that modulates the growth or differentiation of the isolated cranial neural crest stem cell of claim 1, comprising contacting the cell or the population with the agent, and wherein a change of growth or differentiation of the cell or population indicates that the agent modulates the growth or differentiation of the cell or the population.
